(19) United States
(12) Patent Application Publication
(10) Pub. No.: US 2012/0028940 A1
(43) Pub. Date: Feb. 2, 2012

(54) FUNCTIONALLY SELECTIVE AZANITRILE ALPHA-2C ADRENORECEPTOR AGONISTS

(75) Inventors: Christopher W. Boyce, Flemington, NJ (US); Jianhua Chao, San Diego, CA (US); Kevin D. McCormick, Basking Ridge, NJ (US)

(73) Assignee: MERCK SHARP & DOHME CORP., Rahway, NJ (US)

(21) Appl. No.: 13/063,837

(22) PCT Filed: Sep. 15, 2009
(86) PCT No.: PCT/US2009/056967

§ 371 Date: Oct. 24, 2011

Related U.S. Application Data
(60) Provisional application No. 61/097,412, filed on Sep. 16, 2008.

(51) Int. Cl. A61K 31/436; C07D 471/04; A61K 31/437; A61K 31/56; A61P 3/10; A61P 25/00; A61P 11/02; C07D 491/052; A61P 29/00

(52) U.S. Cl. 514/171; 546/115; 546/113; 514/300; 514/302

(57) ABSTRACT

In its many embodiments, the present invention provides a novel class of azanitrile compounds as inhibitors of α2C adrenergic receptor agonists, methods of preparing such compounds, pharmaceutical compositions containing one or more such compounds, methods of preparing pharmaceutical formulations comprising one or more such compounds, and methods of treatment, prevention, inhibition, or amelioration of one or more conditions associated with the α2C adrenergic receptors using such compounds or pharmaceutical compositions.

### RELATED APPLICATIONS

This application claims benefit of provisional application U.S. Ser. No. 61/097,412, filed Sep. 16, 2008, herein incorporated by reference.

### FIELD OF THE INVENTION

The present invention relates to azanitrile compounds useful as α2C adrenergic receptor agonists, methods for making the compounds, pharmaceutical compositions containing the compounds, and methods of treatment and prevention using the compounds and compositions to treat disease states such as congestion (including nasal), migraine, congestive heart failure, cardiac ischemia, glaucoma, stress-induced urinary incontinence, attention deficit disorder, pain and psychotic disorders without substantial adverse side effects associated with α2A receptor agonist treatments.

### BACKGROUND OF THE INVENTION

The initial classification of adrenergic receptors into α- and β-families was first described by Ahlquist in 1948 (Ahlquist R P, “A Study of the Adrenergic Receptors,” Am. J. Physiol. 153:586-600 (1948)). Functionally, the αa-adrenergic receptors were shown to be associated with most of the excitatory functions (vasoconstriction, stimulation of the uterus and pupil dilation). β-adrenergic receptors were implicated in vasodilation, bronchodilation and myocardial stimulation (Lands et al., “Differentiation of Receptor Systems Activated by Sympathomimetic amines,” Nature 214:597-598 (1967)). Since this early work, α-adrenergic receptors have been subdivided into α1- and α2-adrenergic receptors. Cloning and expression of α-adrenergic receptors have confirmed the presence of multiple subtypes of both α1-(α1A, α1B, α1D) and α2-(α2A, α2B, α2C) adrenergic receptors (Michel et al., “Classification of aₗ -Adrenoceptor Subtypes,” Naunyn-Schmiedeberg's Arch. Pharmacol, 352:1-10 (1995); Macdonald et al., “Gene Targeting—Homing in on α₂ -Adrenoceptor-Subtype Function,” TIPS, 18:211-219 (1997)).

Current therapeutic uses of α-2 adrenergic receptor drugs involve the ability of those drugs to mediate many of the physiological actions of the endogenous catecholamines. There are many drugs that act on these receptors to control hypertension, intraocular pressure, eye reddening and nasal congestion and induce analgesia and anesthesia.

α2 adrenergic receptors can be found in the rostral ventrolateral medulla, and are known to respond to the neurotransmitter norepinephrine and the anti hypertensive drug clonidine to decrease sympathetic outflow and reduce arterial blood pressure (Bousquet et al., “Role of the Ventral Surface of the Brain Stem in the Hypothesive Action of Clonidine,” Eur. J. Pharmacol., 34:151-156 (1975); Bousquet et al., ’'Imidazoline Receptors: From Basic Concepts to Recent Developments,” 26:S1-S6 (1995)). Clonidine and other imidazolines also bind to imidazoline receptors (formerly called imidazoline-guanidinium receptive sites or IGRS) (Bousquet et al., “Imidazoline Receptors: From Basic Concepts to Recent Developments,” 26:S1-S6 (1995)). Some researchers have speculated that the central and peripheral effects of imidazolines as hypotensive agents may be related to imidazoline receptors (Bousquet et al., “Imidazoline Receptors: From Basic Concepts to Recent Developments,” 26:S1-S6 (1995); Reis et al., “The lmidazoline Receptor: Pharmacology, Functions, Ligands, and Relevance to Biology and Medicine,” Ann. N.Y. Acad. Sci., 763:1-703 (1995).

Compounds having adrenergic activity are well-known in the art, and are described in numerous patents and scientific publications. It is generally known that adrenergic activity is useful for treating animals of the mammalian species, including humans, for curing or alleviating the symptoms and conditions of numerous diseases and conditions. In other words, it is generally accepted in the art that pharmaceutical compositions having an adrenergic compound or compounds as the active ingredient are useful for treating, among other things, glaucoma, chronic pain, migraines, heart failure, and psychotic disorders.

For example, published PCT application WO 02/076950 discloses compounds having α2 agonist activity of the following general formula:

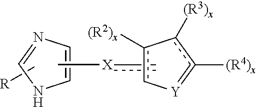

Other publications disclosing similar compounds include WO 01/00586, WO 99/28300, U.S. Pat. No. 6,841,684 B2 and US 2003/0023098 A1.

Another class of compounds having α2-agonist properties is disclosed in U.S. Pat. No. 5,658,938. This class of compounds has the following general formula:

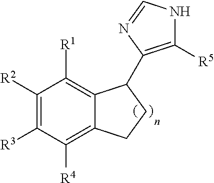

wherein n=1-2. R¹-R³ represent hydrogen, halogen hydroxy, alkyl or alkoxy, and R⁵ is hydrogen or alkyl.

Another class of compounds reported to have affinity for α2 receptors includes the following two compounds (Bagley et.al., Med. Chem. Res. 1994, 4:346-364):

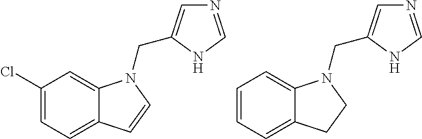

It is also known that compounds having adrenergic activity, such as α2A agonists, may be associated with undesirable side effects. Examples of such side effects include hyper-and hypotension, sedation, locomotor activity, and body temperature variations.

Another class of compounds reported to have affinity for α2 receptors includes the following two compounds (Miller et.al., J. Med. Chem. 1994, 37:2328-2333; J. Med. Chem. 1996, 39:3001-3013; J. Med. Chem. 1997, 37:3014-3024):

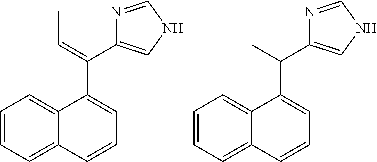

Another class of indane and tetrahyrdonaphthalene type compounds having α2-agonist properties is disclosed in PCT application WO 97/12874 and WO20040506356 This class has the following general formula:

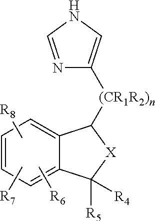

wherein n=0-1, X is 1 or 2 carbon units. R₄ is H, OH, alkyl, or alkoxy, R₅ may be taken together with R⁴ to form a carbonyl, and R⁶-R⁸═H, OH, SH, alkyl, alkenyl, cycloalkyl, alkoxy, hydroxyalkyl, alkylthio, alkylthiol, halo, CF₃, NO₂, or alkylamino. This class specifically includes MPV-2426 (fadolmidine) and its prodrug esters:

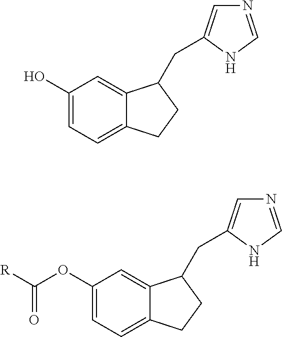

wherein R is optionally substituted lower alkyl, aryl, cycloalkyl, heteroaryl, lower alkylamino, and saturated. 5- or 6-membered heterocyclic groups containing. 1 or 2 N atoms.

Further, other classes of compounds that exhibit functional selectivity for the alpha 2C receptor have been discovered. Application U.S. Ser. No. 11/508,458, filed Aug. 23, 2006 and published as US 2007/0099872, discloses indoline compounds that possess this activity and application U.S. Ser. No. 11/508,467 and published as US 2007/0093477, filed on the same date, describes morpholine compounds that are functionally selective of the alpha 2C receptor. CIP applications of these applications have been filed; the Ser. Nos. are Ser. No. 11/705,673 and published as US 2008/0039439 and Ser. No. 11/705,683 and published as US 2008/0027100, both filed on Feb. 13, 2009.

Additional applications that have been filed by Schering-Plough and disclose alphα2C receptor agonists include applications PCT/US2008/001808; PCT/US2008/001770 and PCT/US2008/001765.

U.S. Pat. No. 6,673,337 describes and claims an ophthalmic composition comprising an alpha-2C agonist component and a solubility enhancing component other than cyclodextrin. The patent does not specifically describe alpha-2C receptor agonists.

Allergan has three published patent applications that describe methylimidazole derivatives that are said to be usedful in treating disease states such as glaucoma, ocular hypertension and congestion. These published applications are: WO 2008/086131 (“Naphthylim)dazoles as Therapeutic agents”); WO 2008/088936 (” Quinolynylmethylimidazoles as Therapuetic Agents“) and WO 2008/088937 (”Qu)nolynylmethylimidazoles as Therapeutic Agents“). WO 2008/088936 discloses the following compound:

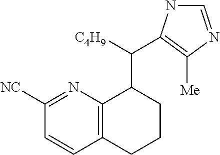

WO 2008/052907 to Hoffmann—La Roche describe substituted 2-imidazoles as modulators of the trace amine associated receptors.

U.S. Pat. No. 5,977,134 to Ciccarone et al. describe peptidomimetic 1,2,3,4-tetrahydroisoquinolines and homologous compounds which inhibit farnesyl-protein transferase (see. Summary of the Invention). Homologues includes 5-imidazolylmethyl indole or 5-imidazolylmethyl indoline derivatives, such as, for example the following compounds:

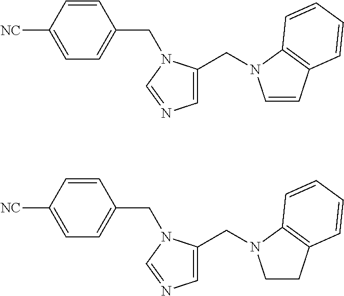

U.S. Pat. No. 7,399,868 to Allegra describes 4-(heteroaryl-methyl and 4-substituted heteroaryl-methyl)-imidazole-2-thiones that are said to act as agonists of the α2C receptor.

It has been discovered in accordance with the present invention that adrenergic compounds that act selectively, and preferably even specifically, as agonists of the α2C or the α2B/α2C (hereinafter referred to as α2C or α2B/2C) receptor subtypes in preference over the α2A receptor subtype and that act functionally selectively as agonists of the α2C or the α2B/2C receptor subtype in preference over the α2A receptor subtype possess desirable therapeutic properties associated with adrenergic receptors but without having one or more undesirable side effects such as changes in blood pressure or sedation. For the purposes of the present invention, a compound is defined to be a specific or at least functionally selective agonist of the α2C receptor subtype over the α2A receptor subtype if the compound's efficacy at the α2C receptor is ≧30% Eₘₐₓ (GTRγS assay) and its efficacy at the α2A receptor is ≦35% Eₘₐₓ (GTPγS assay).

There is a need for new compounds, formulations, treatments and therapies to treat diseases and disorders associated with α2C adrenergic receptors while minimizing adverse side effects. Further, there is a need to develop compounds that are functionally selective for the α2C or the α2B/2C receptor subtype with respect to the α2A receptor subtype. It is, therefore, an object of this invention to provide compounds useful in the treatment or prevention or amelioration of such diseases and disorders.

### SUMMARY OF THE INVENTION

In its many embodiments, the present invention provides a novel class of heterocyclic compounds as functionally selective α2C adrenergic receptor agonists, or metabolites, stereoisomers, salts, solvates or polymorphs thereof, methods of preparing such compounds, pharmaceutical compositions comprising one or more such compounds, methods of preparing pharmaceutical formulations comprising one or more such compounds, and methods of treatment, prevention, inhibition or amelioration of one or more conditions associated with α2C receptors using such compounds or pharmaceutical compositions.

In one aspect, the present application discloses a compound, or pharmaceutically acceptable salts, esters or metabolites, solvates, prodrugs or polymorphs of said compound, said compound having the general structure shown in the Formula:

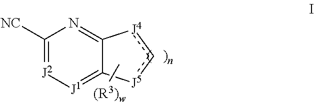

wherein:

J¹, and J² are independently —N—, —N(O)— or —C(R²)—;

J⁴ is:

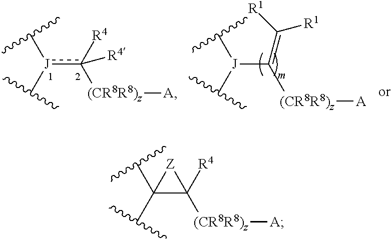

where:
J is —C—. —N—, or —C(R⁶)—;
Z is —[C(R^{a})(R^{a})]ₓ—,
   where
      R^{a} is independently H or alkyl; and
      x is 1, 2, or 3;

J⁵ is —C(R^{6′})—, —N— —N(R^{6′})—, —O— or —S— with the provisio that a double bond is not present between J⁵ and an adjacent ring atom when J⁵ is —O— or —S—;

A is a 5-membered heteroaryl, heterocyclyl or heterocyclenyl ring containing 1-3 heteroatoms (preferably independently selected from the group consisting of oxygen, nitrogen and sulphur);

[symbol] is a single or double bond provided that there cannot be two continuous double bonds and further provided that when atoms 1 and 2 form a double bond, R^{4′} is not present;

R¹ isindependently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one (preferably 1 to 5, more preferably 1 to 3) R⁵;

R² is independently selected from the group consisting of H, —OH, halo, —SF₅, —OSF₅, —CN, —NO₂, —S(O)ₚR⁷, —NR⁷R^{7′}, —(CH₂)_{q}YR^{7′}, —(CH₂)_{q}N(R⁷)YR^{7′}, —(CH₂)_{q}OYR^{7′}, and —(CH₂)_{q}ON═CR⁷R^{7′}, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one (preferably 1 to 5, more preferably 1 to 3) R⁵;

Y is selected from the group consisting of a bond, —C(═O)—, —C(═O)NR⁷—, —C(═O)O—, —C(═NR⁷)—, —C(═NOR⁷)—, —C(═NR⁷)NR⁷—, —C(═NR⁷)NR⁷O—, —S(O)ₚ—, —SO₂NR⁷—, and —C(═S)NR⁷—;

R³ is independently selected from the group consisting of H, halo, and (═O), and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one (preferably 1 to 5, more preferably 1 to 3) R⁵, provided that when w is 3, no more than 2 of the R³ groups may be (═O);

R⁴ is selected from the group consisting of H, —CN, —OH and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one (preferably 1 to 5, more preferably 1 to 3) R⁵;

R^{4′} is absent or selected from the group consisting of H and halo and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one (preferably 1 to 5, more preferably 1 to 3) R⁵;

R⁵ is independently selected from the group consisting of H, halo, —OH, —SF₅, —OSF₅, —CN, —NO₂, —NR⁷R^{7′}, and —S(O)ₚR⁷, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one (preferably 1 to 5, more preferably 1 to 3) of halo, —OH, —CN, —NO₂, —NR⁷R^{7′}, and —S(O)ₚR⁷ substituents and/or 1 or 2 (═O) groups,

R⁶ is independently selected from the group consisting of H, —CN and halo and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one (preferably 1 to 5, more preferably 1 to 3) of halo, —OH, —CN, —NO₂, —NR⁷R^{7′}, and —S(O)ₚR⁷ substituents and/or 1 or 2 (═O) groups, and —C(═O)R⁷, —C(═O)OR⁷, —C(═O)NR⁷R^{7′}, —SO₂R⁷ and —SO₂NR⁷R^{7′};

R^{6′} is independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one (preferably 1 to 5, more preferably 1 to 3) of halo, —OH, —CN, —NO₂, —NR⁷R^{7′}, and —S(O)ₚR⁷ and/or 1 or 2 (═O) groups substituents, and —C(═O)R⁷, —C(═O)OR⁷, —C(═O)NR⁷R^{7′}, —SO₂R⁷ and —SO₂NR⁷R^{7′};

R⁷ is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times (preferably 1 to 5, more preferably 1 to 3) by R¹²;

R^{7′} is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times (preferably 1 to 5, more preferably 1 to 3) by R¹²; or
a) when a variable is —NR⁷R^{7′}, —C(O)NR⁷R^{7′} or —SO₂NR⁷R^{7′}, R⁷ and R^{7′} together with the nitrogen atom to which they are attached independently form a 3- to 8-membered heterocyclyl, heterocyclenyl or heteroaryl ring having, in addition to the N atom, 1 or 2 additional hetero atoms independently selected from the group consisting of O, N, —N(R⁹)— and S, wherein said rings are optionally substituted by 1 to 5 independently selected R¹² moieties and/or 1 or 2 (═O) groups, or b) when a variable is —(CH₂)ₚON═CR⁷R^{7′}, R⁷ and R^{7′} together with the carbon atom to which they are attached independently form a 3- to 8-membered cycloalkyl, cycloalkenyl, aryl, heterocyclyl, heterocyclenyl or heteroaryl ring, wherein said heterocyclyl, heterocyclenyl or heteroaryl rings have 1-3 heteroatoms which are independenyly selected from the group consisting of O, N, —N(R⁹)— and S, wherein said rings are optionally substituted by 1 to 5 independently selected R¹² moieties and/or 1 or 2 (═O) groups,

R⁸ is independently selected from the group consisting H, alkyl, halo, nitrile, and alkoxy;

R⁹ is independently selected from the group consisting of H, —C(O)—R¹⁰, —C(O)—OR¹⁰, and —S(O)ₚ—R¹⁰ and alkyl, alkenyl, alkynyl, cycloalkyl, aryl, arylalkyl, heteroaryl, and heteroarylalkyl groups, each of which is optionally substituted with at least one (preferably 1 to 5, more preferably 1 to 3) of halo, —OH, —CN, —NO₂, —N(R¹¹)₂, and —S(O)ₚR¹¹ substituents and/or 1 or 2 (═O) groups; and

R¹⁰ is independently selected from the group consisting of alkyl, alkenyl, alkynyl, cycloalkyl, aryl, arylalkyl, heteroaryl, and heteroarylalkyl groups, each of which is optionally substituted with at least one (preferably 1 to 5, more preferably 1 to 3) of halo, —OH, —CN, —NO₂, —N(R¹¹)₂, and —S(O)ₚR¹¹ substituents and/or 1 or 2 (═O) groups;

R¹¹ is a moiety independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl, each of which is optionally substituted by at least one (preferably 1 to 5, more preferably 1 to 3) substituent independently selected from the group consisting of halo, —OH, —CN, —NO₂, —N(R^{11′})₂, and —S(O)ₚR^{11′} and/or 1 or 2 (═O) groups;

R^{11′} is independently selected from the group consisting of H, alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl;

R¹² is independently selected from selected from the group consisting of H, halo, —OH, —CN, —NO₂, —N(R¹¹)₂, —C(O)—OR¹⁴, —N(R¹⁴)—C(O)—R¹⁴, —N(R¹⁴)—C(O)₂—R¹⁴, —C(O)—N(R¹¹)₂ , —N(R¹⁴)—S(O)₂—R^{11′}, —S(O)₂—N(R¹¹)₂ and —S(O)ₚR¹¹ and/or 1 or 2 (═O) groups, and alkyl, alkoxy, alkenyl, alkenyloxy, alkynyl, cycloalkyl, cycloalkenyl, cycloalkoxy, aryl, aryloxy, arylalkyl, heteroaryl, heteroaryloxy, heteroarylalkyl, heterocyclyl, heterocyclenyl, heterocyclenyloxy, heterocyclylalkyl, heterocyclenylalkyl, arylalkoxy, heteroarylalkoxy, heterocyclylalkoxy, and heterocyclenylalkoxy groups, each of which in turn is optionally substituted by at least once (preferably 1 to 5, more preferably 1 to 3) by a substituent selected from the group consisting of H, alkyl, haloalkyl, halo, —OH, optionally substituted alkoxy, optionally substituted aryloxy, optionally substituted cycloalkoxy, optionally substituted heteroaryloxy, optionally substituted heterocyclenyloxy, —CN, —NO₂, —N(R¹¹)₂, and —S(O)ₚR¹¹ and/or 1 or 2 (═O) groups, wherein said optionally substituted alkoxy, aryloxy, optionally substituted cycloalkoxy, optionally substituted heteroaryloxy, and heterocyclenyloxy when substituted are substituted one or more (preferably 1 to 5, more preferably 1 to 3) times by R¹¹;

R¹⁴ is independently H, alkyl, or aryl;

m is 0 or 1;

n is independently 1, 2, or 3;

p is independently 0, 1, or 2;

q is independently an integer from 0 to 6;

w is 0, 1, 2, 3, 4, or 5; and

z is 0, 1, 2, 3, 4, or 5;

with the following provisos:

(a) if J is N, then J⁵ is —C(R^{6′})—;

(b) if J⁵ is —O—, —S—, —N—, or —N(R^{6′})—, then J is —C— or —O(R⁶)—; and

(c) if J⁵ is —N(R^{6′})—, then [symbol] cannot be a double bond between atoms 1 and 2 in J⁴.

The compounds of Formula I can be useful as α2C adrenergic receptor agonists, and can be useful in the treatment and prevention of allergic rhinitis, congestion (including, but not limited to nasal congestion), migraine, congestive heart failure, cardiac ischemia, glaucoma, stress-induced urinary incontence, attention deficit hyperactivity disorder, neuronal damage from ischemia and psychotic disorders. Further, the compounds of Formula I can be useful in the treatment of pain (both chronic and acute), such as pain that is caused by inflammation, neuropathy, arthritis (including osteo and rheumatoid arthritis), diabetes (e.g., diabetes mellitus or diabetes insipidus) or pain of an unknown origin. Examples of neuropathic pain may include but not limited to; diabetic neuropathy, neuralgia of any etiology (e.g. post-herpetic, trigeminal), chemotherapy-induced neuropathy, HIV, lower back pain of neuropathic origin (e.g. sciatica), traumatic peripheral nerve injury of any etiology, central pain (e.g. post-stroke, thalamic, spinal nerve injury). Other pain that can be treated is nociceptive pain and pain that is visceral in origin or pain that is secondary to inflammation or nerve damage in other diseases or diseases of unknown origin. Further, the compounds of Formula I can be useful in the treatment of symptoms of diabetes. Examples of symptoms of diabetes may include but are not limited to: hyperglycemia, hypertriglyceridemia, increased levels of blood insulin and hyperlipidemia.

Alternatively, the present invention provides for a method for the treatment of congestion in a mammal in need thereof which comprises administering to a mammal an effective dose of at least one compound having adrenergic activity wherein said compound is a functionally selective agonist of the α2c receptor.

A further embodiment of the present invention is a method for the treatment of congestion in a mammal in need thereof which comprises administering to a mammal an effective dose of at least one compound having adrenergic activity wherein said compound is a functionally selective agonist of the α2C receptor, wherein the selective agonist of the α2c receptor has an efficacy that is greater than or equal to 30% Eₘₐₓ when assayed in the GTPγS assay and its efficacy at the α2A receptor is ≦35% Eₘₐₓ (GTRγS assay).

Another embodiment of the present invention is a method for the treatment of congestion in a mammal in need thereof without modifying the blood pressure at therapeutic doses which comprises administering to the mammal an effective dose of at least one compound having adrenergic activity wherein said compound is a selective agonist of the α2C receptor.

### DETAILED DESCRIPTION

In an embodiment, the present invention discloses certain heterocyclic compounds which are represented by structural Formula I, or a pharmaceutically acceptable salt or solvate thereof, wherein the various moieties are as described above.

In one embodiment, the present invention discloses compounds of Formula Ia which are represented by the structural Formula Ia

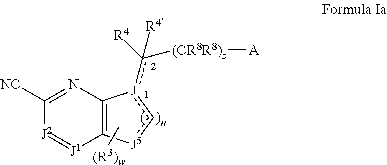

or a pharmaceutically acceptable salt, ester, solvate or prodrug thereof, wherein:

A is a 5-membered heteroaryl, heterocyclyl or heterocyclenyl ring containing 1-3 heteroatoms;

J¹ and J² are independently —N—, —N(O)— or —C(R²)—;

J is C, N, or —C(R⁶)—;

J⁵ is —C(R^{6′})—, —N—, —N(R^{6′})—, —O— or —S— with the proviso that a double bond is not present between J⁵ and an adjacent ring atom when J⁵ is —O— or —S—;

[symbol] is a single or double bond provided that there cannot be two continuous double bonds and further provided that when atoms 1 and 2 form a double bond, R^{4′} is not present;

R² is independently selected from the group consisting of H, —OH, halo, —SF₅, —OSF₅, —CN, —NO₂, —S(O)ₚR⁷, —NR⁷R^{7′}, —(CH₂)ₚYR⁷, —(CH₂)_{q}N(R⁷)YR^{7′}, —(CH₂)_{q}OYR^{7′}, and —(CH₂)ₚON═CR⁷R^{7′}, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

Y is selected from the group consisting of a bond, —C(═O)—, —C(═O)NR⁷—, —C(═O)O—, —C(═NR⁷)—, —C(═NOR⁷)—, —C(═NR⁷)NR⁷—, —C(═NR⁷)NR⁷O—, —S(O)ₚ——SO₂NR⁷—, and —C(═S)NR⁷;

R³ is independently selected from the group consisting of H, halo, and (═O), and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵, provided that when w is 3, no more than 2 of the R³ groups may be (═O);

R⁴ is selected from the group consisting of H, —CN, —OH and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

R^{4′} is absent or selected from the group consisting of H and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

R⁵ is independently selected from the group consisting of H, halo, —OH, —SF₅, OSF₅, —CN, —NO₂, —NR⁷R^{7′}, and —S(O)ₚR⁷, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ substituents and/or 1 or 2 (═O) groups,

R⁶ is independently selected from the group consisting of H, —CN and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚa⁷substituents and/or 1 or 2 (═O) groups, and —C(═O)R⁷, —C(═O)OR⁷, —C(═O)NR⁷R^{7′}, —SO₂R⁷ and —SO₂NR⁷R^{7′};

R⁶′ is independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ and/or 1 or 2 (═O) groups substituents, and —C(═O)R⁷, —C(═O)OR⁷, —C(═O)NR⁷R^{7′}, —SO₂R⁷ and —SO₂NR⁷R^{7′};

R⁷ is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times by R¹²;

R^{7′} is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times by R¹²; or
a) when a variable is —NR⁷R^{7′}, —C(O)NR⁷R^{7′} or —SO₂NR⁷R^{7′}, R⁷ and R^{7′} together with the nitrogen atom to which they are attached independently form a 3- to 8-membered heterocyclyl, heterocyclenyl or heteroaryl ring having, in addition to the N atom, 1 or 2 additional hetero atoms independently selected from the group consisting of O, N, —N(R⁹)— and S, wherein said rings are optionally substituted by 1 to 5 independently selected R¹² moieties and/or 1 or 2 (═O) groups, or
b) when a variable is —(CH₂)_{q}ON═CR⁷R^{7′}, R⁷ and R^{7′} together with the carbon atom to which they are attached independently form a 3- to 8-membered cycloalkyl, cycloalkenyl, aryl, heterocyclyl, heterocyclenyl or heteroaryl ring, wherein said heterocyclyl, heterocyclenyl or heteroaryl rings have 1-3 heteroatoms which are independently selected from the group consisting of O, N, —N(R⁹)— and S, wherein said rings are optionally substituted by 1 to 5 independently selected R¹² moieties and/or 1 or 2 (═O) groups,

R⁸ is independently selected from the group consisting H, alkyl, halo, nitrile, and alkoxy;

R⁹ is independently selected from the group consisting of H, —C(O)—R¹⁰, —C(O)—OR¹⁰, and —S(O)ₚ—R¹⁰ and alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents and/or 1 or 2 (═O) groups; and

R¹⁰ is independently selected from the group consisting of alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents and/or 1 or 2 (═O) groups;

R¹¹ is a moiety independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl, each of which is optionally substituted by at least one substituent independently selected from the group consisting of halo, —OH, —CN, —NO₂, —N(R^{11′})₂, and —S(O)ₚR^{11′} and/or 1 or 2 (═O) groups;

R^{11′} is independently selected from the group consisting of H, alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl;

R¹² is independently selected from selected from the group consisting of H, halo, —OH, —CN, —NO₂, —N(R¹¹)₂, —C(O)—OR¹⁴, —N(R¹⁴)—C(O)—R¹⁴, —N(R¹⁴)—C(O)₂—R¹⁴, —C(O)—N(R¹¹)₂, —N(R¹⁴)—S(O)₂—R^{11′}, —S(O)₂—N(R¹¹)₂ and —S(O)ₚR¹¹ and/or 1 or 2 (═O) groups, and alkyl, alkoxy, alkenyl, alkenyloxy, alkynyl, cycloalkyl, cycloalkenyl, cycloalkoxy, aryl, aryloxy, arylalkyl, heteroaryl, heteroaryloxy, heteroarylalkyl, heterocyclyl, heterocyclenyl, heterocyclenyloxy, heterocyclylalkyl, heterocyclenylalkyl, arylalkoxy, heteroarylalkoxy, heterocyclylalkoxy, and heterocyclenylalkoxy groups, each of which in turn is optionally substituted by at least once by a substituent selected from the group consisting of H, alkyl, haloalkyl, halo, —OH, optionally substituted alkoxy, optionally substituted aryloxy, optionally substituted cycloalkoxy, optionally substituted heteroaryloxy, optionally substituted heterocyclenyloxy, —CN, —NO₂, —N(R¹¹)₂, and —S(O)ₚR¹¹ and/or 1 or 2 (═O) groups, wherein said optionally substituted alkoxy, aryloxy, optionally substituted cycloalkoxy, optionally substituted heteroaryloxy, and heterocyclenyloxy when substituted are substituted one or more times by R¹¹;

R¹⁴ is independently H, alkyl, or aryl;

n is independently 1, 2 or 3;

p is independently 0-2;

q is independently an integer from 0-6;

w is 0, 1, 2, 3, 4, or 5; and

z is 0, 1, 2, 3, 4, or 5,

with the following provisos:

(a) if J is N, then J⁵ is —C(R^{6′})—;

(b) if J⁵ is O, S—N—or —N(R^{6′})—, then J is —C— or —C(R⁶)—; and

(c) if J⁵ is —N(R^{6′})—, then [symbol] cannot be a double bond between atoms 1 and 2 in J⁴.

In another embodiment, the present invention discloses compounds of Formula I which are represented by the structural Formula Ib

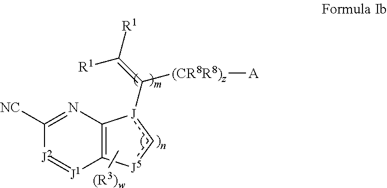

or a pharmaceutically acceptable salt, ester, solvate or prodrug thereof, wherein:

A is a 5-membered heteroaryl, heterocyclyl or heterocyclenyl ring containing 1-3 heteroatoms;

J¹, and J² are independently —N—, —N(O)— or —C(R²)—;

J is C, N, or —C(R⁶)—;

J⁵ is —C(R^{6′})—, —N—, —N(R^{6′})—, —O— or —S—, with the proviso that a double bond is not present between J⁵ and an adjacent ring atom when J⁵ is —O— or —S—;

[symbol] is a single or double bond provided that there cannot be two continuous double bonds;

R¹ is independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

R² is independently selected from the group consisting of H, —OH, halo, —SF₅, —OSF₅, —CN, —NO₂, —S(O)ₚR⁷, —NR⁷R^{7′}, —(CH₂)_{q}YR^{7′}, —(CH₂)_{q}N(R⁷)YR^{7′}, —(CH₂)_{q}OYR^{7′}, and —(CH₂)_{q}ON═CR⁷R^{7′}, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

Y is selected from the group consisting of a bond, —C(═O)—, —C(═O)NR⁷—, —C(═O)O—, —C(═NR⁷)—, —C(═NOR⁷)—, —C(═NR⁷)NR⁷—, —C(═NR⁷)NR⁷O—, —S(O)ₚ—, —SO₂NR⁷—, and —C(═S)NR⁷—;

R³ is independently selected from the group consisting of H, halo, and (═O), and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵, provided that when w is 3, no more than 2 of the R³ groups may be (═O);

R⁵ is independently selected from the group consisting of H, halo, —OH, —SF₅, —OSF₅, —CN, —NO₂, —NR⁷R^{7′}, and —S(O)ₚR⁷, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ substituents and/or 1 or 2 (═O) groups,

R⁶ is independently selected from the group consisting of H, —CN, and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ substituents and/or 1 or 2 (═O) groups, and —C(═O)R⁷, —C(═O)OR⁷, —C(═O)NR⁷R^{7′}, —SO₂R⁷ and —SO₂NR⁷R^{7′};

R^{6′} is independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ and/or 1 or 2 (═O) groups substituents, and —C(═O)R⁷, —C(═O)OR⁷, —C(═O)NR⁷R^{7′}, —SO₂R⁷ and —SO₂NR⁷R^{7′};

R⁷ is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times by R¹²;

R^{7′} is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times by R¹²; or
a) when a variable is —NR⁷R^{7′}, —C(O)NR⁷R^{7′} or —SO₂NR⁷R^{7′}, R⁷ and R^{7′} together with the nitrogen atom to which they are attached independently form a 3- to 8-membered heterocyclyl, heterocyclenyl or heteroaryl ring having, in addition to the N atom, 1 or 2 additional hetero atoms independently selected from the group consisting of O, N, —N(R⁹)— and S, wherein said rings are optionally substituted by 1 to 5 independently selected R¹² moieties and/or 1 or 2 (═O) groups, or
b) when a variable is —(CH₂)ₚON═CR⁷R^{7′}, R⁷ and R^{7′} together with the carbon atom to which they are attached independently form a 3- to 8-membered cycloalkyl, cycloalkenyl, aryl, heterocyclyl, heterocyclenyl or heteroaryl ring, wherein said heterocyclyl, heterocyclenyl or heteroaryl rings have 1-3 heteroatoms which are independently selected from the group consisting of O, N, —N(R⁹)— and S, wherein said rings are optionally substituted by 1 to 5 independently selected R¹² moieties and/or 1 or 2 (═O) groups,

R⁸ is independently selected from the group consisting H, alkyl, halo, nitrile, and alkoxy;

R⁹ is independently selected from the group consisting of H, —C(O)—R¹⁰, —C(O)—OR¹⁰, and —S(O)ₚ—R¹⁰ and alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents and/or 1 or 2 (═O) groups; and

R¹⁰ is independently selected from the group consisting of alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents and/or 1 or 2 (═O) groups;

R¹¹ is a moiety independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl, each of which is optionally substituted by at least one substituent independently selected from the group consisting of halo, —OH, —CN, —NO₂, —N(R^{11′})₂, and —S(O)ₚR^{11′} and/or 1 or 2 (═O) groups;

R^{11′} is independently selected from the group consisting of H, alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl;

R¹² is independently selected from selected from the group consisting of H, halo, —OH, —CN, —NO₂, —N(R¹¹)₂, —C(O)—OR¹⁴, —N(R¹⁴)—C(O)—R¹⁴, —N(R¹⁴)—C(O)₂—R¹⁴, —C(O)—N(R¹¹)₂, —N(R¹⁴)—S(O)₂—R^{11′}, —S(O)₂—N(R¹¹)₂ and —S(O)ₚR¹¹ and/or 1 or 2 (═O) groups, and alkyl, alkoxy, alkenyl, alkenyloxy, alkynyl, cycloalkyl, cycloalkenyl, cycloalkoxy, aryl, aryloxy, arylalkyl, heteroaryl, heteroaryloxy, heteroarylalkyl, heterocyclyl, heterocyclenyl, heterocyclenyloxy, heterocyclylalkyl, heterocyclenylalkyl, arylalkoxy, heteroarylalkoxy, heterocyclylalkoxy, and heterocyclenylalkoxy groups, each of which in turn is optionally substituted by at least once by a substituent selected from the group consisting of H, alkyl, haloalkyl, halo, —OH, optionally substituted alkoxy, optionally substituted aryloxy, optionally substituted cycloalkoxy, optionally substituted heteroaryloxy, optionally substituted heterocyclenyloxy, —CN, —NO₂, —N(R¹¹)₂, and —S(O)ₚR¹¹ and/or 1 or 2 (═O) groups, wherein said optionally substituted alkoxy, aryloxy, optionally substituted cycloalkoxy, optionally substituted heteroaryloxy, and heterocyclenyloxy when substituted are substituted one or more times by R¹¹;

R¹⁴ is independently H, alkyl, or aryl;

m is 0 or 1;

n is independently 1, 2, or 3;

p is independently 0, 1, or 2;

q is independently an integer from 0-6;

w is 0, 1, 2, 3, 4, or 5; and

z is 0, 1, 2, 3, 4, or 5

with the following provisos:

(a) if J is N, then J⁵ is —C(R^{6′})—; and

(b) if J⁵ is O, S, —N— or —N(R^{6′})—, then J is —C— or —C(R⁶)—.

In another embodiment, the present invention discloses compounds of Formula I which is represented by the structural Formula Ic

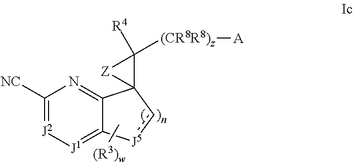

or a pharmaceutically acceptable salt, ester, solvate or prodrug thereof, wherein:

A is a 5-membered heteroaryl, heterocyclyl or heterocyclenyl ring containing 1-3 heteroatoms;

J¹, and J² are independently —N—, —N(O)— or —C(R²)—;

J⁵ is —C(R^{6′})—, —N—, —N(R^{6′})—, —O— or —S—, with the proviso that a double bond is not present between J⁵ and an adjavent ring atom when J⁵ is —O— or —S—;

[symbol] is a single or double bond provided that there cannot be two continuous double bonds;

R² is independently selected from the group consisting of H, —OH, halo, —SF₅, OSF₅, —CN, —NO₂, —S(O)ₚR⁷, —NR⁷R^{7′}, —(CH₂)_{q}YR^{7′}, —(CH₂)_{q}N(R⁷)YR^{7′}, —(CH₂)_{q}OYR⁷, and —(CH₂)_{q}ON═CR⁷R^{7′}, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

Y is selected from the group consisting of a bond, —C(═O)—, —C(═O)NR⁷—, —C(═O)O—, —C(═NR⁷)—, —C(═NOR⁷)—, —C(═NR⁷)NR⁷—, —C(═NR⁷)NR⁷O—, —S(O)ₚ—, —SO₂NR⁷—, and —C(═S)NR⁷—;

R³ is independently selected from the group consisting of H, halo, and (═O), and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵, provided that when w is 3, no more than 2 of the R³ groups may be (═O);

R⁴ is selected from the group consisting of H, —CN, —OH and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

R⁵ is independently selected from the group consisting of H, halo, —OH, —SF₅, OSF₅, —CN, —NO₂, —NR⁷R^{7′}, and —S(O)ₚR⁷, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ substituents and/or 1 or 2 (═O) groups,

R^{6′} is independently selected from the group consisting of H, —CN and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ and/or 1 or 2 (═O) groups substituents, and —C(═O)R⁷, —C(═O)OR⁷, —C(═O)NR⁷R^{7′}, —SO₂R⁷ and —SO₂NR⁷R^{7′};

R⁷ is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times by R¹²;

R^{7′} is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times by R¹²; or
a) when a variable is —NR⁷R^{7′}, —C(O)NR⁷R^{7′} or —SO₂NR⁷R^{7′}, R⁷ and R^{7′} together with the nitrogen atom to which they are attached independently form a 3- to 8-membered heterocyclyl, heterocyclenyl or heteroaryl ring having, in addition to the N atom, 1 or 2 additional hetero atoms independently selected from the group consisting of O, N, —N(R⁹)— and S, wherein said rings are optionally substituted by 1 to 5 independently selected R¹² moieties and/or 1 or 2 (═O) groups, or
b) when a variable is —(CH₂)_{q}ON═CR⁷R^{7′}, R⁷ and R^{7′} together with the carbon atom to which they are attached independently form a 3- to 8-membered cycloalkyl, cycloalkenyl, aryl, heterocyclyl, heterocyclenyl or heteroaryl ring, wherein said heterocyclyl, heterocyclenyl or heteroaryl rings have 1-3 heteroatoms which are independently selected from the group consisting of O, N, —N(R⁹)— and S, wherein said rings are optionally substituted by 1 to 5 independently selected R¹² moieties and/or 1 or 2 (═O) groups,

R⁸ is independently selected from the group consisting H, alkyl, halo, nitrile, and alkoxy;

R⁹ is independently selected from the group consisting of H, —C(O)—R¹⁰, —C(O)—OR¹⁰, and —S(O)ₚ—R¹⁰ and alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents and/or 1 or 2 (═O) groups; and

R¹⁰ is independently selected from the group consisting of alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents and/or 1 or 2 (═O) groups;

R¹¹ is a moiety independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl, each of which is optionally substituted by at least one substituent independently selected from the group consisting of halo, —OH, —CN, —NO₂, —N(R^{11′})₂, and —S(O)ₚR^{11′} and/or 1 or 2 (═O) groups;

R^{11′} is independently selected from the group consisting of H, alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl;

R¹² is independently selected from selected from the group consisting of H, halo, —OH, —CN, —NO₂, —N(R¹¹)², —C(O)—OR¹⁴, —N(R¹⁴)—C(O)—R¹⁴, —N(R¹⁴)—C(O)₂—R¹⁴, —C(O)—N(R¹¹)₂, —N(R¹⁴)—S(O)₂—R^{11′}, —and/or 1 or 2 (═O) groups, and alkyl, alkoxy, alkenyl, alkenyloxy, alkynyl, cycloalkyl, cycloalkenyl, cycloalkoxy, aryl, aryloxy, arylalkyl, heteroaryl, heteroaryloxy, heteroarylalkyl, heterocyclyl, heterocyclenyl, heterocyclenyloxy, heterocyclylalkyl, heterocyclenylalkyl, arylalkoxy, heteroarylalkoxy, heterocyclylalkoxy, and heterocyclenylalkoxy groups, each of which in turn is optionally substituted by at least once by a substituent selected from the group consisting of H, alkyl, haloalkyl, halo, —OH, optionally substituted alkoxy, optionally substituted aryloxy, optionally substituted cycloalkoxy, optionally substituted heteroaryloxy, optionally substituted heterocyclenyloxy, —CN, —NO₂, —N(R¹¹)₂, and —S(O)ₚR¹¹ and/or 1 or 2 (═O) groups, wherein said optionally substituted alkoxy, aryloxy, optionally substituted cycloalkoxy, optionally substituted heteroaryloxy, and heterocyclenyloxy when substituted are substituted one or more times by R¹¹;

R¹⁴ is independently H, alkyl, or aryl;

n is independently 1, 2, or 3;

p is independently 0, 1, or 2;

q is independently an integer from 0-6;

w is 0, 1, 2, 3, 4, or 5; and

z is 0, 1, 2, 3, 4, or 5,

with the following proviso:

(a) if J⁵ is —O—, —S— or —N(R^{6′})—, then J is —C— or —C(R⁶)—.

In one embodiment A is a 5-membered heteroaryl, heterocyclenyl or heterocyclyl ring. Preferred heteroaryl, heterocyclenyl or heterocyclyl 5-membered rings include, for example, imidazole, thiazole, pyrrole, isoxazole, oxazole, isothiazole, pyrazole, imadazoline, imidazol-2-one, imidazol-2-thione, 2-aminoimidazoline, oxazoline, oxazol-2-one, oxazol-2-thione, 2-aminooxazoline, thiazoline, thiazol-2-one, thiazol-2-thione, 2-aminothiazoline, pyrroline, pyrazoline, pyrrolidine, imidazolidine, and pyrazolidine. A more preferred set of 5-membered rings includes: imidazole, imadazoline, imidazol-2-one, imidazol-2-thione, 2-aminoimidazoline, oxazoline, oxazol-2-one, oxazol-2-thione, and 2-aminooxazoline. A most preferred 5-membered ring is imidazole.

In another embodiment, if J¹ and J² are —C(H)— and A is imidazolyl.

In another embodiment, if J¹ and J² are —C(H)—, J is —N—, J⁵ is —C(H)—, n is 1, R³ is H, R⁴ is H, R⁴′ is H, A is imidazolyl.

In another embodiment, if J¹ and J² are —C(H)—, J is —C—, J⁵ is —N(R^{6′})—, n is 1 or 2, R⁴ is H, R⁴′ is H, A is imidazolyl, z=0.

In another embodiment, if J¹ and J² are —C(H)—, J is —N—, J⁵ is —C(H)— and J⁵ with an atom adjacent to it forms a double bond, n is 1, R³ is H, R⁴ is H, R⁴′ is H, A is imidazolyl.

In another embodiment, if J¹ and J² are —C(H)—, J is —C—, J⁵ is —O—, n is 1 or 2, R⁴ is H, R⁴′ is H, A is imidazolyl, z=0, and n=1 or 2.

In another embodiment, if J¹ and J² are —C(H)—, J is —C—, J⁵ is —S—, n is 1, R³ is H, R⁴ is H, R⁴′ is H and z=0.

In another embodiment, if J is N, then J⁵ is —C(R^{6′})—.

In another embodiment, if J is C, then J⁵ is —N(R^{6′})—

In another embodiment, if J is C, then J⁵ is —O—.

In another embodiment, if J is C, then J⁵ is —S—.

In another embodiment, if J is C, then J⁵ is —C(R^{6′})—.

In another embodiment, if J is C(R⁶), then J⁵ is —O—.

In another embodiment, if J is C(R⁶), then J⁵ is —S—.

In another embodiment, if J is C(R⁶), then J⁵ is —C(R^{6′})—.

In another embodiment, J¹ and J² are each —C(R²)—.

In another embodiment, J¹ is —N—.

In another embodiment, J² is —N—.

In another embodiment, J¹ and J²are both —N—.

In another embodiment, A is a 5-membered heterocyclic ring containing at least one ring nitrogen.

In another embodiment R¹ is independently H or alkyl.

In another embodiment, R² is independently selected from the group consisting of H, —OH, halo, —CN, —NO₂—S(O)R⁷, —NR⁷R^{7′}, —(CH₂)_{q}YR^{7′}, —(CH₂)ₚN(R⁷)YR^{7′}, —(CH₂)ₚOYR^{7′}, and —(CH₂)ₚON═CR⁷R^{7′}, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵.

In another embodiment, R² is H.

In another embodiment, Y is selected from a bond, —C(═O)—, —C(═O)NR⁷—, —C(═O)O—, —S(O)ₚ—, and —SO₂NR⁷—.

In another embodiment, R³ is indepenently selected from H, —CN and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵.

In another embodiment, R⁴ is independently selected from H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵.

In another embodiment, R^{4′} is independently selected from H and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵.

In another embodiment, in Formula la, atoms 1 and 2 form a double bond and R^{4′} does not exist.

In another embodiment, R⁵ is independently selected from H, halo, —OH, —CN, —NO₂, —NR⁷R^{7′}, and —S(O)ₚR⁷, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ substituents.

In another embodiment, R⁵ is independently selected from H, halo, —OH, —CN, and alkyl.

In another embodiment, R⁶ is independently selected from H and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ substituents, and —C(═O)R⁷, —C(═O)OR⁷, —C(═O)NR⁷R^{7′}, —SO₂R⁷ and —SO₂—NR⁷R^{7′}.

In another embodiment, R^{6′} is independently selected from H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ substituents, and —C(═O)R⁷, —C(═O)OR⁷, —C(═O)NR⁷R^{7′}, —SO₂R⁷ and —SO₂—NR⁷R^{7′}.

In another embodiment, R^{6′} is independently selected from H, optionally substituted alkyl, —C(═O)R⁷, —C(═O)OR⁷, —C(═O)NR⁷R^{7′}, —SO₂R⁷ and —NR⁷R^{7′}.

In another embodiment, R⁷ is independently selected from H and alkyl, alkenyl, alkynyl, cycloalkyl, aryl, arylalkyl, heterocyclyl, heteroaryl, and heteroarylalkyl groups, each of which is optionally substituted with at least one of halo, alkoxy, —OH, —CN, —NO₂, —N(R¹¹)₂, and —S(O)ₚR¹¹ substituents.

In another embodiment, R^{7′} is independently selected from H and alkyl, alkenyl, alkynyl, cycloalkyl, aryl, arylalkyl, heterocyclyl, heteroaryl, and heteroarylalkyl groups, each of which is optionally substituted with at least one of halo, alkoxy, —OH, —CN, —NO₂, —N(R¹¹)₂, and —S(O)ₚR¹¹ substituents.

In another embodiment, when a variable is —NR⁷R^{7′}, —C(O)NR⁷R^{7′}or —SO₂NR⁷R^{7′}, R⁷ and R^{7′} together with the N atom to which they are attached form a aziridine, azetidine, pyrrole, pyrrolidine, piperidine, piperazine or morpholine ring, each of which are optionally substituted by R¹².

In another embodiment, R⁸ is independently selected from H, halo or alkyl.

In another embodiment, R¹² is independently alkyl, haloalkyl, —NO₂, —CN, halo, —OH, amino, alkylamino, dialkylamino or alkoxy.

In another embodiment, m is 1 and z is 0-5, more preferably 0-3, most preferably 0 or 1.

In another embodiment, n is 1.

In another embodiment, n is 2.

In another embodiment, p is 0-2.

In another embodiment, z is an interger from 0-6, preferably 1-5, most preferably 1-3.

In another embodiment, m is 0 and z is an integer from 1-5, preferably 1 or 2, most preferably 1.

In another embodiment, J is N.

In another embodiment, J⁵ is —N(R⁶′)—.

In another embodiment, J⁵ is —N—.

In another embodiment, the present invention discloses compounds which are represented by structural formulae II-IX or a pharmaceutically acceptable salt, solvate or ester thereof, wherein the various definitions are those described above for Formula I:

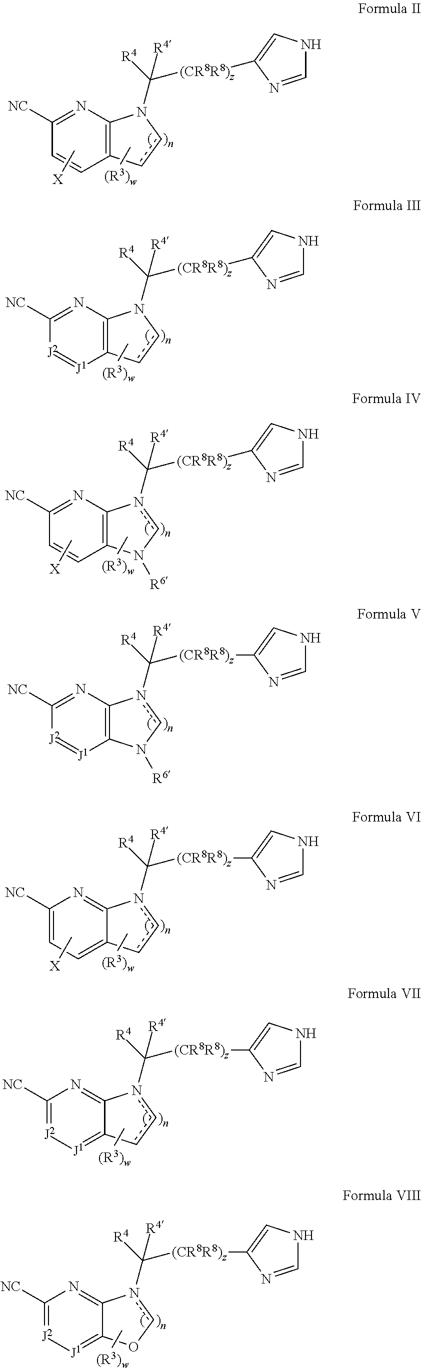

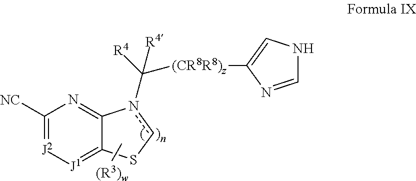

Another embodiment of the compounds of Formulae II-IX are those wherein
X is halo or H.
J¹, and J² are independently —N— or —C(R²)—,
n is 1 or 2,
z is an interger from 1 to 3, and
   the remaining definitions are defined above in Formula I.

Another embodiment of the present invention is compounds of Formulae II or III of the formuale

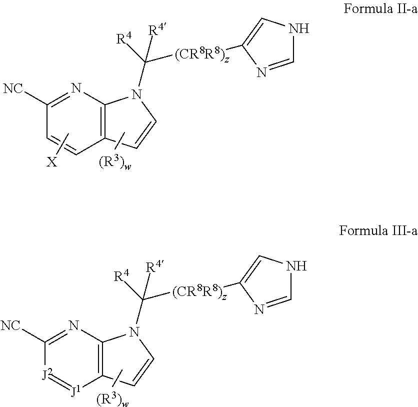

wherein z is 0 or 1, w is 0 or 1, R⁴ is H and the remaining definitions are defined above in Formula I.

Another embodiment of the present invention is the compounds of Formula I that have the structural formula IV

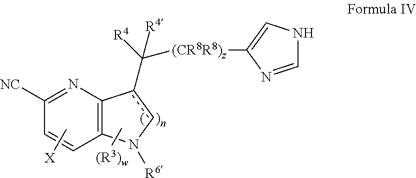

or a pharmaceutically acceptable salt, ester, solvate, or prodrug of said compound, wherein:

X is H or halo;

[symbol] is a single or double bond;

R³ is independently selected from the group consisting of H and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

R⁴ is independently selected from the group consisting of H, —CN, —OH and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

R⁵ is independently selected from the group consisting of H, halo, —OH, —CN, —NO₂, —NR⁷R^{7′}, and —S(O)ₚR⁷, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ substituents and/or 1 or 2 (═O) groups;

R⁶′ is independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ and/or 1 or 2 (═O) groups substituents, and —C(═O)R⁷, —C(═O)OR⁷, —C(═O)NR⁷R^{7′}, —SO₂R⁷ and —SO₂NR⁷R^{7′};

R⁷ is independently selected from the group consisting of H and alkyl, alkenyl, alkynyl, cycloalkyl, aryl, arylalkyl, heterocyclyl, heteroaryl, and heteroarylalkyl groups, each of which is optionally substituted with at least one of halo, alkoxy, —OH, —CN, —NO₂, —N(R¹¹)₂ and —S(O)ₚR¹¹ substituents;

R^{7′} is independently selected from the group consisting of of H and alkyl, alkenyl, alkynyl, cycloalkyl, aryl, arylalkyl, heterocyclyl, heteroaryl, and heteroarylalkyl groups, each of which is optionally substituted with at least one of halo, alkoxy, —OH, —CN, —NO₂, —N(R¹¹)₂ and —S(O)ₚR¹¹ substituents ; or
a) when a variable is is —NR⁷R^{7′}, —C(O)NR⁷R^{7′} or —SO₂NR⁷R^{7′}, R⁷ and R^{7′} together with the nitrogen atom to which they are attached form a 3- to 8-membered heterocyclyl, heterocyclenyl or heteroaryl ring having, in addition to the N atom, 1 or 2 additional hetero atoms selected from the group consisting of O, N, —N(R⁹)— and S, wherein said rings are optionally substituted by 1 to 5 independently selected R¹² moieties,

R⁸ is independently H or alkyl;

R⁹ is independently selected from the group consisting of H, —C(O)—R¹⁰, —C(O)—OR¹⁰, and —S(O)ₚ—R¹⁰ and alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents; and

R¹⁰ is selected from the group consisting of alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents;

R¹¹ is a moiety independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl;

z is an integer from 0-5;

n is independently 1, 2, or 3;

p is independently 0, 1 or 2;

q is independently an integer from 0-6; and

w is 0, 1, 2, or 3.

Another embodiment of the present invention is the compounds of Formula I that have the structural formula V

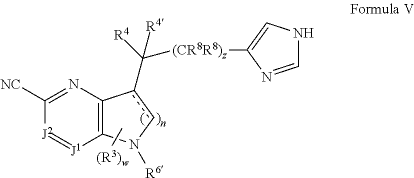

or a pharmaceutically acceptable salt, ester, solvate or prodrug of said compound, wherein:

J¹, and J² are independently —N— or —C(R²)—;

[symbol] is a single or double bond;

R² is independently selected from the group consisting of H, —OH, halo, —CN, —NO₂, —S(O)ₚR⁷, —NR⁷R^{7′}, —(CH₂)_{q}YR^{7′}, —(CH₂)_{q}N(R⁷)YR^{7′}, —(CH₂)_{q}OYR^{7′}, and —(CH₂)_{q}ON═CR⁷R^{7′}, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

Y is selected from a bond, —C(═O)—, —C(═O)NR⁷—, —C(═O)O—, —S(O)ₚ—, and —SO₂NR⁷—.

R³ is independently selected from the group consisting of H, and halo, and (═O), and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵ provided that when w is 3, no more than 2 of the R³ groups may be (═O);

R⁴ is independently selected from the group consisting of H and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

R⁵ is independently selected from the group consisting of H, halo, —OH, —CN, —NO₂, —NR⁷R^{7′}, and —S(O)ₚR⁷, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ substituents and/or 1 or 2 (═O) groups;

R^{6′} is independently selected from the group consisting of H and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ substituents, and —C(═O)R⁷, —C(═O)OR⁷, —C(═O)NR⁷R^{7′}, —SO₂R⁷ and —SO₂NR⁷R^{7′};

R⁷ is independently selected from the group consisting of H and alkyl, alkenyl, alkynyl, cycloalkyl, aryl, arylalkyl, heterocyclyl, heteroaryl, and heteroarylalkyl groups, each of which is optionally substituted with at least one of alkyl, haloalkyl, halo, alkoxy, —OH, —CN, —NO₂, —N(R¹¹)₂ and —S(O)ₚR¹¹ substituents;

R^{7′} is independently selected from the group consisting of H and alkyl, alkenyl, alkynyl, cycloalkyl, aryl, arylalkyl, heterocyclyl, heteroaryl, and heteroarylalkyl groups, each of which is optionally substituted with at least one of alkyl, haloalkyl, halo, alkoxy, —OH, —CN, —NO₂, —N(R¹¹)₂ and —S(O)ₚR¹¹ substituents; or
a) when a variable is is —NR⁷R⁷,^{′}—C(O)NR⁷R^{7′} or —SO₂NR⁷R^{7′}, R⁷ and R^{7′} together with the nitrogen atom to which they are attached form a 3- to 8-membered heterocyclyl, heterocyclenyl or heteroaryl ring having, in addition to the N atom, 1 or 2 additional hetero atoms selected from the group consisting of O, N, —N(R⁹)— and S, wherein said rings are optionally substituted by 1 to 5 independently selected R¹² moieties,

R⁸ is independently H or alkyl;

R⁹ is independently selected from the group consisting of H, —C(O)—R¹⁰, —C(O)—OR¹⁰, and —S(O)ₚ—R¹⁰ and alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents; and

R¹⁰ is selected from the group consisting of alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents;

R¹¹ is a moiety independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl;

z is an integer from 0-5;

n is independently 1, 2, or 3;

p is independently 0, 1, or 2;

q is independently an integer from 0-6; and

w is 0, 1, 2, or 3.

Another embodiment of the present invention is the compounds of Formula I that have the structural formula VI

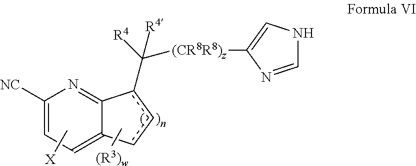

wherein
X is H or halo;
z is is an interger from 0-5;
w is is an integer from 0-3; and
n is independently 1, 2 or 3,
   or a pharmaceutically acceptable salt, ester, solvate or prodrug thereof.

Another embodiment of the present invention are the compounds of Formula I that have the structural Formula VII

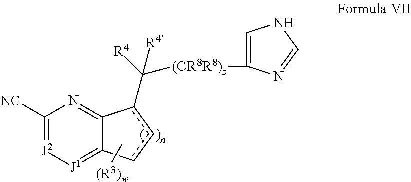

wherein:

J¹ and J² are independently —N— or —(CR²)—;
z is an integer from 0-5;
w is 0, 1, 2, or 3; and
n is independently 1, 2, or 3,
   or a pharmaceutically acceptable salt, ester, solvate or prodrug thereof.

Another embodiment of the compounds of Formula I is compounds represented by the structural Formula X

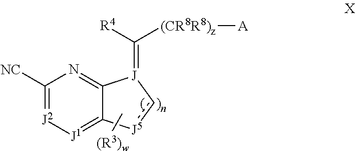

or a pharmaceutically acceptable salt, ester, solvate or prodrug thereof, wherein:

A is a 5-membered heteroaryl, heterocyclyl or heterocyclenyl ring containing 1-3 heteroatoms;

J¹ and J² are independently —N—, —N(O)— or —C(R²)—;

J is C;

J⁵ is —C(R^{6′})—, —N—, —N(R^{6′})—, —O— or —S—, with the proviso that a double bond is not present between J⁵ and an adjaent ring atom when J⁵; is —O— or —S—;

[symbol] is a single or double bond provided that there cannot be two continuous double bonds

R² is independently selected from the group consisting of H, —OH, halo, —SF₅, OSF₅, —CN, —NO₂, —S(O)ₚR⁷, —NR⁷R^{7′}, —(CH₂)_{q}YR^{7′}, —(CH₂)_{q}N(R⁷)YR^{7′}, —(CH₂)_{q}OYR^{7′}, and —(CH₂)_{q}ON═CR⁷R^{7′}, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

Y is selected from the group consisting of a bond, —C(═O)—, —C(═O)NR⁷—, —C(═O)O—, —C(═NR⁷)—, —C(═NOR⁷)—, —C(═NR⁷)NR⁷—, —C(═NR⁷)NR⁷O—, —S(O)ₚ—, —SO₂NR⁷—, and —C(═S)NR⁷—;

R³ is independently selected from the group consisting of H, halo, and (═O), and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵, provided that when w is 3, no more than 2 of the R³ groups may be (═O);

R⁴ is selected from the group consisting of H, —CN, and halo, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups optionally substituted with at least one R⁵;

R⁵ is independently selected from the group consisting of H, halo, —OH, —SF₅, —OSF₅, —SF₅, —OSF₅, —CN, —NO₂, —NR⁷R^{7′}, and —S(O)ₚR⁷, and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ substituents and/or 1 or 2 (═O) groups,

R^{6′} is independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl groups, each of which is optionally substituted with at least one of halo, —OH, —CN, —NO₂, —NR⁷R^{7′}, and —S(O)ₚR⁷ and/or 1 or 2 (═O) groups substituents, and —C(═O)R⁷, —C(═O)OR⁷, —C(═O)NR⁷R^{7′}, —SO₂R⁷ and —SO₂NR⁷R^{7′};

R⁷ is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times by R¹²;

R^{7′} is independently selected from the group consisting of H and alkyl, alkenyl, alkynyl, cycloalkyl, cycloalkylalkyl, cycloclenyl, cyclocyclenylalkyl, aryl, arylalkyl, heterocyclyl, heterocyclylalkyl, hetrocyclenyl, hetrocyclenylalkyl, heteroaryl, and heteroarylalkyl groups, each of which is optionally substituted one or more times by R¹²; or
a) when a variable is —NR⁷R^{7′}, —C(O)NR⁷R^{7′} or —SO₂NR⁷R^{7′}, R⁷ and R^{7′} together with the nitrogen atom to which they are attached independently form a 3- to 8-membered heterocyclyl, heterocyclenyl or heteroaryl ring having, in addition to the N atom, 1 or 2 additional hetero atoms independently selected from the group consisting of O, N, —N(R⁹)— and S, wherein said rings are optionally substituted by 1 to 5 independently selected R¹² moieties and/or 1 or 2 (═O) groups, or
b) when a variable is —(CH₂)_{q}ON═CR⁷R^{7′}, R⁷ and R^{7′} together with the carbon atom to which they are attached independently form a 3- to 8-membered cycloalkyl, cycloalkenyl, aryl, heterocyclyl, heterocyclenyl or heteroaryl ring, wherein said heterocyclyl, heterocyclenyl or heteroaryl rings have 1-3 heteroatoms which are independently selected from the group consisting of O, N, —N(R⁹)— and S, wherein said rings are optionally substituted by 1 to 5 independently selected R¹² moieties and/or 1 or 2 (═O) groups,

R⁸ is independently selected from the group consisting H, alkyl, halo, nitrile, and alkoxy;

R⁹ is independently selected from the group consisting of H, —C(O)—R¹⁰, —C(O)—OR¹⁰, and —S(O)ₚ—R¹⁰ and alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents and/or 1 or 2 (═O) groups; and

R¹⁰ is independently selected from the group consisting of alkyl, alkenyl, alkynyl, cycloalkyl, aryl, arylalkyl, heteroaryl, and heteroarylalkyl groups, each of which is optionally substituted with at least one of halo, —OH, —CN, —NO₂, —N(R¹¹)₂, and —S(O)ₚR¹¹ substituents and/or 1 or 2 (═O) groups;

R¹¹ is a moiety independently selected from the group consisting of H and alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl, each of which is optionally substituted by at least one substituent independently selected from the group consisting of halo, —OH, —CN, —NO₂, —N(R^{11′})₂, and —S(O)ₚR^{11′} and/or 1 or 2 (═O) groups;

R^{11′} is independently selected from the group consisting of H, alkyl, alkoxy, alkenyl, alkenyloxy, alkynyl, cycloalkyl, cycloalkoxy, aryl, aryloxy, arylalkyl, heteroaryl, heteroarylalkyl, heterocyclyl, and heterocyclylalkyl;

R¹² is independently selected from selected from the group consisting of H, halo, —OH, —CN, —NO₂, —N(R¹¹)₂, —C(O)—OR¹⁴, —N(R¹⁴)—C(O)—R¹⁴, —N(R¹⁴)—C(O)₂—R¹⁴, —C(O)—N(R¹¹)₂, —N(R¹⁴)—S(O)₂—R^{11′}, —S(O)₂—N(R¹¹)₂ and —S(O)ₚR¹¹ and/or 1 or 2 (═O) groups, and alkyl, alkoxy, alkenyl, alkenyloxy, alkynyl, cycloalkyl, cycloalkenyl, cycloalkoxy, aryl, aryloxy, arylalkyl, heteroaryl, heteroaryloxy, heteroarylalkyl, heterocyclyl, heterocyclenyl, heterocyclenyloxy, heterocyclylalkyl, heterocyclenylalkyl, arylalkoxy, heteroarylalkoxy, heterocyclylalkoxy, and heterocyclenylalkoxy groups, each of which in turn is optionally substituted by at least once by a substituent selected from the group consisting of H, alkyl, haloalkyl, halo, —OH, optionally substituted alkoxy, optionally substituted aryloxy, optionally substituted cycloalkoxy, optionally substituted heteroaryloxy, optionally substituted heterocyclenyloxy, —CN, —NO₂, —N(R¹¹)₂, and —S(O)ₚR¹¹ and/or 1 or 2 (═O) groups, wherein said optionally substituted alkoxy, aryloxy, optionally substituted cycloalkoxy, optionally substituted heteroaryloxy, and heterocyclenyloxy when substituted are substituted one or more times by R¹¹;

R¹⁴ is independently H, alkyl, or aryl;

n is independently 1, 2, or 3;

p is independently 0, 1, or 2;

q is independently an integer from 0-6;

w is 0, 1, 2 ,3 ,4 or 5; and

z is 0, 1, 2, 3, 4, or 5

with the following provisos:

(a) if J is N, then J⁵ is —C(R^{6′})—; and

(b) if J⁵ is O, S, —N— or —N(R^{6′})—, then J is —C— or —C(R⁶)—.

An embodiment of the compounds of Formula X is compounds represented by structural Formual Xa:

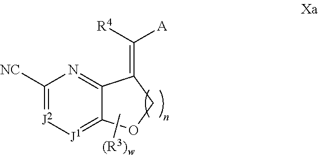

or their pharmaceutically acceptable esters or salts, wherein J¹ and J² are —C(R²)— the variables are those defined above for Formula X.

A further embodiment of the compounds of Formula Xa or their pharmaceutically acceptable esters or salts wherein:

A is imidazole;

R² is independently H, —OH, halo, cyano, nitro, —S(O)—R⁷ or —N(R⁷)(R^{7′});

n is 2;

w is 0 or 1; and

R³ is independently H or alkyl.

A further embodiment of the compounds of Formula X is compounds represented by structural Formula Xb

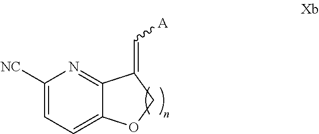

or a pharmaceutically acceptable ester of salt thereof whererin

n is 1 or 2; and

[symbol] indicates that A may be cis or trans with the bicyclic ring.

A further embodiment of the compounds of Formula X is compounds represented by structural Formual XI

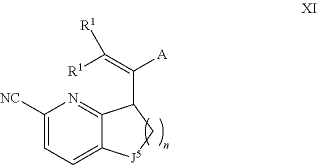

or a pharmaceutically acceptable ester or salt thereof

wherein

A is imidazole;

n is 1 or 2; and

J⁵ is —(CH₂)—, —O—, or —S—.

A group of compounds is shown below:

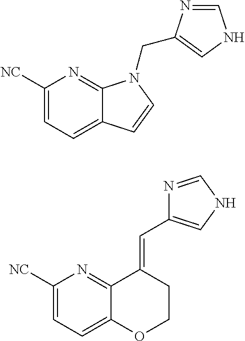

A further embodiment of the present invention is compounds of Formula I in isolated and purified form.

Another embodiment of the present invention is a method for selectively stimulating α2C adrenergic receptors in a cell in need thereof, comprising contacting said cell with a therapeutically effective amount of at least one compound of Formula I.

As used above, and throughout this disclosure, the following terms, unless otherwise indicated, shall be understood to have the following meanings:

“Patient” includes both human and animals.

“Mammal” means humans and other mammalian animals.

“Congestion” refers to all type of congestion including, but not limited to, congestion associated with perennial allergic rhinitis, seasonal allergic rhinitis, non-allergic rhinitis, vasomotor rhinitis, rhinitis medicamentosa, sinusitis, acute rhinosinusitis, or chronic rhinosinusitis or when the congestion is caused by polyps or is associated with the common cold.

“Alkyl” means an aliphatic hydrocarbon group which may be straight or branched and comprising about 1 to about 20 carbon atoms in the chain.

Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. The term “substituted alkyl” means that the alkyl group may be substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. The term “substituted alkynyl” means that the alkynyl group may be substituted by one or more substituents which may be the same or different, each substituent being independently selected from the group consisting of alkyl, aryl and cycloalkyl.

“Aryl” means an aromatic monocyclic or multicyclic ring system, in which at least one of the multicyclic rings is an aryl ring, comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

Non-limiting examples of aryl multicyclic ring systems include:

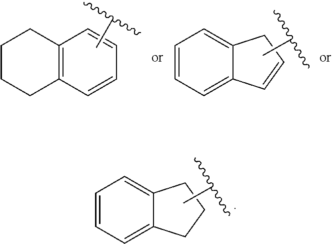

“Heteroaryl” means an aromatic monocyclic or multicyclic ring system, in which at least one of the multicyclic rings is aromatic, comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-bithiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

Non-limiting examples of heteroaryl multicyclic ring systems systems include:

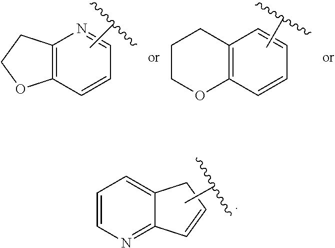

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Halogen” and “Halo” mean fluorine, chlorine, bromine, or iodine. Preferred are fluorine, chlorine or bromine, and more preferred are fluorine and chlorine.

“Ring system substituent” means a substituent attached to an aromatic or non-aromatic ring system which, for example, replaces an available hydrogen on the ring system. Ring system substituents may be the same or different, each being independently selected from the group consisting of aryl, heteroaryl, aralkyl, alkylaryl, heteroaralkyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, Y₁Y₂N—, Y₁Y₂N-alkyl-, Y₁Y₂NC(O)— and Y₁Y₂NSO₂—, wherein Y₁ and Y₂ may be the same or different and are independently selected from the group consisting of hydrogen, alkyl, aryl, and aralkyl.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protected moieties are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, imidazolidinyl, pyrazolidinyl and the like.

Compounds of Formula I and salts, esters, solvates and prodrugs thereof, may exist in their tautomeric form (for example, as an amide or imino ether). All such tautomeric forms are contemplated herein as part of the present invention. Non-limiting examples of tautomeric forms that are part of this invention are as follows:

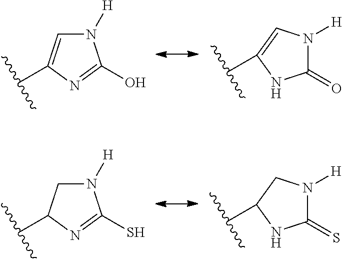

It should be noted that in saturated heterocyclyl containing systems of this invention, there are no hydroxyl, amino, or thiol groups on carbon atoms adjacent to a N, O or S atom. Thus, for example, in the ring:

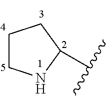

there is no —OH attached directly to carbons marked 2 and 5. It should also be noted that this definition does not preclude (═O), (═S), or (═N) substitutions, or their tautomeric forms, on C atoms adjacent to a N, O or S. Thus, for example, in the above ring, (═O) substitution on carbon 5, or its imino ether tautomer is allowed.

Non-limiting examples which illustrate the present invention are as follows:

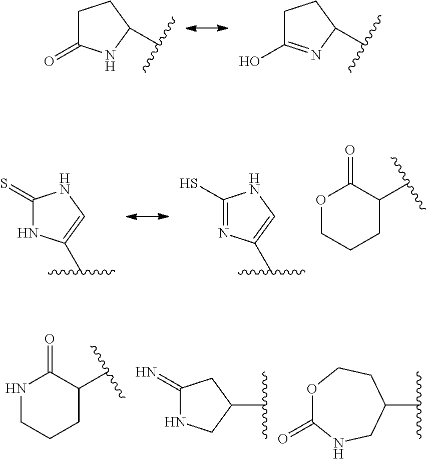

The following non-limiting examples serve to illustrate radicals not contemplated by the present invention:

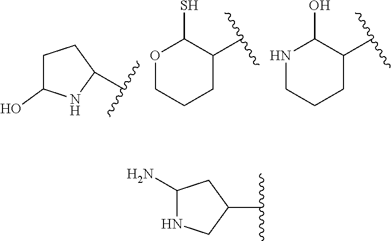

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Heterocyclylalkyl” or “heteroarylalkyl” means a heterocyclyl-alkyl group in which the heterocyclyl and the alkyl are as previously described. Preferred heterocyclylalkyls contain a lower alkyl group. Non-limiting examples of suitable heterocyclylalkyl groups include piperidylnnethyl, piperidylethyl, pyrrolidylmethyl, nnorpholinylpropyl, piperazinylethyl, azindylnnethyl, azetidylethyl, oxiranylpropyl and the like. The bond to the parent moiety is through the alkyl group.

“Heterocyclenyl” (or “heterocycloalkeneyl”) means a non-aromatic nnonocyclic or nnulticyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide. S-oxide or S,S-dioxide. Non-limiting examples of suitable monocyclic azaheterocyclenyl groups include 1,2,3,4-tetrahydropyridyl, 1,2-dihydropyridyl, 1,4-dihydropyridyl, 1,2,3,6-tetrahydropyridyl, 1,4,5,6-tetrahydropyrin1idyl, 2-pyrrolinyl, 3-pyrrolinyl, 2-imidazolinyl, 2-pyrazolinyl, 2-oxazolinyl, 2-thiazolinyl, and the like. Non-limiting examples of suitable oxaheterocyclenyl groups include 3,4-dihydro-2H-pyran, dihydrofuranyl, fluorodihydrofuranyl, and the like. Non-limiting example of a suitable multicyclic oxaheterocyclenyl group is 7-oxabicyclo[2.2.1]heptenyl. Non-linniting examples of suitable monocyclic thiaheterocyclenyl rings include dihydrothiophenyl, dihydrothiopyranyl, and the like.

“Heterocyclenylalkyl” means a heterocyclenyl-alkyl group in which the heterocyclenyl and the alkyl are as previously described.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an organic acid group in which the —OH of the carboxyl group is replaced by some other substituent. Suitable non-limiting examples include H—C(O)—, alkyl-C(O)—, cycloalkyl-C(O)—, heterocyclyl-C(O)—, and heteroaryl-C(O)— groups in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” or “arylalkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Heteroarylalkoxy” means a heteroarylalkyl-O-group in which the heteroarylalkyl group is as previously described.

“Heterocyclylalkoxy” means a heterocyclylalkyl-O group in which the hetrocyclylalkyl group is as previously described.

“Heterocyclenylalkoxy” means a heterocyclenylalkyl-O group in which the heterocyclenylalkyl group is as previously described.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound' or “stable structure” is meant a compound that is sufficiently robust to survive isolation to a useful degree of purity from a reaction mixture, and formulation into an efficacious therapeutic agent.

It is noted that carbons of formula I can be replaced with 1-3 silicon atoms, provided all valency requirements are satisfied.

The term “optionally substituted” means optional substitution with the specified groups, radicals or moieties.

The straight line — as a bond generally indicates a mixture of, or either of, the possible isomers, non-limiting example(s) include, containing (R)— and (S)— stereochemistry. For example,

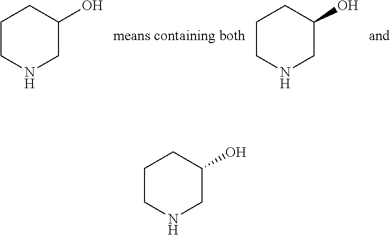

A dashed line ( [symbol] ) represents an optional bond.

Lines drawn into the ring systems, such as, for example:

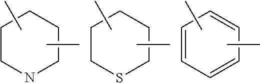

indicate that the indicated line (bond) may be attached to any of the substitutable ring atoms, non-limiting examples include carbon, nitrogen and sulfur ring atoms.

As well known in the art, a bond drawn from a particular atom wherein no moiety is depicted at the terminal end of the bond indicates a methyl group bound through that bond to the atom, unless stated otherwise. For example:

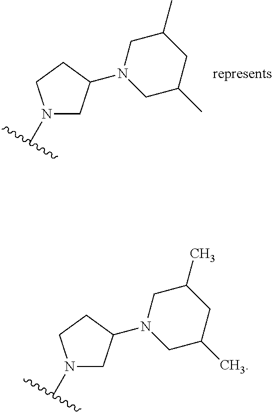

It should also be noted that any heteroatom with unsatisfied valences in the text, schemes, examples and Tables herein is assumed to have the hydrogen atom to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or formula, its definition on each occurrence is independent of its definition at every other occurrence.

Unless defined otherwise, all definitions for the variables follow the convention that the group to the right forms the point of attachement to the molecule; i.e., if a definition is arylalkyl, this means that the alkyl portion of the definition is attached to the molecule.

Further, all divalent variable are attached from left to right. For example when R² is —(CH₂)_{q}N(R⁷)YR^{7′}, and Y is —C(═O)NR⁷—, then R² forms the group —(CH₂)_{q}N(R⁷)—C(═O)N(R⁷)—R^{7′}.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of formula I or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

For example, if a compound of Formula I or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as 8-dimethylaminoethyl), carbamoyl-(C₁-C₂)alkyl, N,N-di(C₁-C₂)alkylcarbamoyl-(C1-C2)alkyl and piperidino-, pyrrolidino- or morpholino(C₂-C₃)alkyl, and the like.

Similarly, if a compound of Formula I contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula I incorporates —NH— functional group, such as in a primary or secondary amine or in a nitrogen-containing heterocycle, such as imidazole or piperazine ring, a prodrug can be formed by the replacement of a hydrogen atom in the amine group with a group such as, for example, R-carbonyl, RO-carbonyl, NRR'-carbonyl where R and R′ are each independently (C₁-C₁₀)alkyl, (C₃-C₇)cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N—or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of illustrative solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

Metabolic conjugates, such as glucuronides and sulfates which can undergo reversible conversion to the compounds of Formula I are contemplated in the present invention.

“Effective amount” or “therapeutically effective amount” is meant to describe an amount of compound or a composition of the present invention effective in producing the desired therapeutic, ameliorative, inhibitory or preventative effect.

The terms “purified”, “in purified form” or “in isolated and purified form,” as used herein, for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like) , in sufficient purity to be characterizable by standard analytical techniques described herein or well known to the skilled artisan.

“Capsule” is meant to describe a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

“Tablet” is meant to describe a compressed or molded solid dosage form containing the active ingredients with suitable diluents. The tablet can be prepared by compression of mixtures or granulations obtained by wet granulation, dry granulation or by compaction.

“Oral gels” is meant to describe to the active ingredients dispersed or solubilized in a hydrophillic semi-solid matrix.

“Powders for constitution” refers to powder blends containing the active ingredients and suitable diluents which can be suspended in water or juices.

“Diluent” refers to substances that usually make up the major portion of the composition or dosage form. Suitable diluents include sugars such as lactose, sucrose, mannitol and sorbitol; starches derived from wheat, corn, rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

“Disintegrants” refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

“Binders” refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

“Lubricant” is meant to describe a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d'l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

“Glidents” means materials that prevent caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

“Coloring agents” refers to excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

“Bioavailability” refers to the rate and extent to which the active drug ingredient or therapeutic moiety is absorbed into the systemic circulation from an administered dosage form as compared to a standard or control. Conventional methods for preparing tablets are known. Such methods include dry methods such as direct compression and compression of granulation produced by compaction, or wet methods or other special procedures. Conventional methods for making other forms for administration such as, for example, capsules, suppositories and the like are also well known.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons or sulfurs on various substituents, including enantiomeric forms (which may exist even in the absence of asymmetric carbons), rotameric forms, atropisomers, and diastereomeric forms, are contemplated within the scope of this invention. For example, if a compound of Formula I incorporates a double bond or a fused ring, both the cis- and trans-forms, as well as mixtures, are embraced within the scope of the invention. Individual stereoisomers of the compounds of the invention may, for ex ample, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, racemates or prodrugs of the inventive compounds.

Diasteromeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diasteromeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formulae Ia and Ib may be atropisomers (e.g., substituted biaryls) and are considered as part of this invention. Enantiomers can also be separated by use of chiral HPLC column.

Polymorphic forms of the compounds of Formula I, and of the salts, solvates and prodrugs of the compounds of Formula I, are intended to be included in the present invention

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³O, ¹⁴O, ¹⁵N, ¹⁸O, ¹⁷O, ³¹P, ³²P, ³⁸S, ¹⁸F, and ³⁶Cl, respectively.

Certain isotopically-labelled compounds of Formula I (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples hereinbelow, by substituting an appropriate isotopically labelled reagent for a non-isotopically labelled reagent.

The compounds according to the invention have pharmacological properties; in particular, the compounds of Formula I can be useful as α2C adrenoreceptor agonists.

A preferred dosage is about 0.001 to 500 mg/kg of body weight/day of the compound of Formula I. An especially preferred dosage is about 0.01 to 25 mg/kg of body weight/day of a compound of Formula I, or a pharmaceutically acceptable salt or solvate of said compound.

The compounds of this invention may also be useful in combination (administered together or sequentially) with one or more therapeutic agents such as, for example, glucosteroids. PDE-4 inhibitors, anti-muscarinic agents, cromolyn sodium, H₁ receptor antagonists, 5-HT₁ agonists, NSAIDs, angiotensin-converting enzyme inhibitors, angiotensin II receptor agonists, β-blockers, β-agonists (including both long and short acting), leukotriene antagonists, diuretics, aldosterone antagonists, ionotropic agents, natriuretic peptides, pain management/analgesic agents, anti-anxiety agents, anti-migraine agents, and therapeutic agents suitable for treating heart conditions, psychotic disorders, and glaucoma.

Suitable steroids include prednisolone, fluticasone (including all ester such as the propionate or furoate esters), triamcinolone, beclomethasone, mometasone (including any ester form such as mometasone furoate), budasamine, ciclesonide betamethasone, dexamethasone, prednisone, flunisolide, and cortisone.

Suitable PDE-4 inhibitors include roflumilast, theophylline, rolipram, piclamilast, cilomilast and CDP-840.

Suitable antiimuscarinic agents include ipratropium bromide and tiatropium bromide.

Suitable H₁ antagonists include astemizole, azatadine, azelastine, acrivastine, brompheniramine, cetirizine, chlorpheniramine, clemastine, cyclizine, carebastine, cyproheptadine, carbinoxamine, descarboethoxyloratidine, diphenhydramine, doxylamine, dimethindene, ebastine, epinastine, efletirizeine, fexofenadine, hydroxyzine, ketotifen, loratidine, levocabastine, meclizine, fexofenadine, hydroxyzine, ketotifen, loratadine, levocabastine, meclizine, mizolastine, mequitazine, mianserin, noberastine, norastemizole, picumast, pyrilamine, promethazine, terfenadine, tripelennamine, temelastine, trimeprazine or triprolidine.

Suitable anti-inflammatory agents include aspirin, diclofenac, diflunisal, etodolac, flurbiprofen, ibuprofen, indomethacin, ketoprofen, ketorolac, nabumetone, naproxen, oxaprozin, piroxicam, sulindac, and tolmetin.

Suitable aldosterone antagonists include spironolactone. Suitable ionotropic agents include digitalis.

Suitable angiotensin II receptor agonists include irbesartan and losartan.

Suitable diuretics include spironolactone, methyclothiazide, bumetanide, torsemide, hydroflumethiazide, trichlormethiazide, hydroclorothiazide, triamterene, ethacrynic acid, methyclothiazide, hydrochlorothiazide, benzthiazide, hydrochlorothiazide, quinethazone, hydrochlorothiazide, chlorthalidone, furosemide, indapamide, hydroclorothiazide, triamterene, trichlormethiazide, hydrochlorothiazide, amiloride HCl, amiloride HCl, metolazone, trichlormethiazide, bendroflumethiazide, hydrochlorothiazide, polythiazide, hydroflumethiazide, chlorthalidone, and metolazone.

Suitable pain management/analgesic agents include Celecoxib, amitriptyline, ibuprofen, naproxen, gabapentin, tramadol, rofecoxib, oxycodone HCl, acetaminophenoxycodone HCl, carbamazepine, amitriptyline, diclofenac, diclofenac, etodolac, fenoprofen calcium, flurbiprofen, ibuprofen, indomethacin, ketoprofen, ketorolac tromethamine, mefenamic acid, meloxicam, nabumetone, naproxen, oxaprozin, piroxicam, sulindac, tolmetin sodium, valdecoxib, diclofenac/misoprostol, oxycontin, vicodin, darvocet, percocet, morphine sulfate, dilaudid, stadol, stadol NS, acetaminophen with codeine, acetaminophen with codeine #4, Lidoderm® patches, ziconotide, duloxetine, roboxetine, gabapentin and pregabalin.

Suitable β-blockers include acebutolol, atenolol, atenolol/chlorthalidone, betaxolol, bisoprolol fumarate, bisoprolol/HCTZ, labetolol, metoprolol tartrate, nadolol, pindolol, propranolol, propranolol/HCTZ, sotalol, and timolol.

Suitable β-agonists include dobutamine, ritodrine, salbutamol, levalbuterol, metaproternol, formoterol, fenoterol, bambuterol, brocaterol, clenbuterol, terbutaline, tulobuterol, epinephrine, isoprenalin, and hexoprenalin.

Suitable leucotriene antagonists include levamisole.

Suitable anti-migraine agents include rovatriptan succinate, naratriptan HCl, rizatriptan benzoate, sumatriptan succinate, zolmitriptan, almotriptan malate, methysergide maleate, dihydroergotamine mesylate, ergotamine tartrate, ergotamine tartrate/caffeine, Fioricet®, Fiorninal®, Depakene®, and Depakote®.

Suitable anti-anxiety and anti-depressant agents include amitriptyline HCl, bupropion HCl, citalopram hydrobromide, clomipramine HCl, desipramine, fluoxetine, fluvoxamine maleate, maprotiline HCl, mirtazapine, nefazodone HCl, nortriptyline, paroxetine HCl, protriptyline HCl, sertraline HCl, doxepin, and trimipramine maleate.

Suitable angiotensin converting enzyme inhibitors include Captopril, enalapril, enalapril/HCTZ, lisinopril, lisinopril/HCTZ, and Aceon®.

The pharmacological properties of the compounds of this invention may be confirmed by a number of pharmacological assays. The exemplified pharmacological assays which are described later have been carried out with the compounds according to the invention and their salts.

This invention is also directed to pharmaceutical compositions which comprise at least one compound of Formula I or a pharmaceutically acceptable salt or solvate of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. When preparing a liquid preparation, the inclusion of one or more solubility enhancing components is excluded. Solubility enhancing components are described, for example, in U.S. Pat. No. 6,673,337 in column 2, line 50 to column 3, line 17 and in column 6, line 49 to line 31; U.S. Pat. No. 6,673,337 is expressly incorporated by reference. Specific solubility enhancing agents that are excluded in the liquid form preparations include metal carboxymethylcelluloses, metal carboxymethylhydroxyethylcelloses, hydroxypropylmethyl celluloses derivative of these compounds, and cyclodextrins. As an example may be mentioned water or water-propylene glycol solutions for parenteral injection or addition of sweeteners and opacifiers for oral solutions, suspensions and emulsions. Liquid form preparations may also include solutions or suspensions for intranasal administration.

An aspect of this invention is that the pharmaceutical composition is in a solid dosage form comprising a compound of Formula I or a pharmaceutical acceptable salt, ester, solvate or prodrug thereof and a least one pharmaceutically acceptable carrier, adjuvant or vehicle.

Another aspect of this invention is a liquid, aqueous pharmaceutical composition is comprising a compound of Formula I or a pharmaceutical acceptable salt, ester, solvate or prodrug thereof and a least one pharmaceutically acceptable carrier, adjuvant or vehicle provided that the adjuvant is not a solubility enhancing component, such as those described in U.S. Pat. No. 6,673,337 (discussed above).

Another aspect of this invention is a liquid, aqueous pharmaceutical composition is comprising a compound of Formula I or a pharmaceutical acceptable salt, ester, solvate or prodrug thereof and a least one pharmaceutically acceptable carrier, adjuvant or vehicle wherein if a solubility enhancement component is present it is cyclodextrin.

Another aspect of this invention is a pharmaceutical formulation that is a nasal spray wherein the pH is equal to or less that about 6.5, more preferably between about 6.1 to 6.2.

Another aspect of this invention the formulation is a nasal spray wherein the adjuvants include a suspending agent (e.g., AVICEL (such as AVICIL RC-581, RC-591 and CL-611), which are microcrystalline cellulose and carboxymethylcellulose sodium; hydroxypropylmethyl cellulose; methyl cellulose; polyvinyl alcohol; or CARBOPOL) and a humectant (e.g., glycerin, propylene glycol; polyethylene glycol; povidone; or dextrose).

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions or suspensions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula I or a pharmaceutically acceptable salt or solvate of said compound and a pharmaceutically acceptable carrier, vehicle or diluent.

Yet another aspect of this invention is a kit comprising an amount of at least one compound of Formula I, or a pharmaceutically acceptable salt or solvate of said compound and an amount of at least one therapeutic agent listed above, wherein the amounts of the two or more ingredients result in desired therapeutic effect.

In general, the compounds in the invention may be produced by a variety of processes know to those skilled in the art and by know processes analogous thereto. The invention disclosed herein is exemplified by the following preparations and examples which should not be construed to limit the scope of the disclosure. Alternative mechanistic pathways and analogous structures will be apparent to those skilled in the art. The practitioner is not limited to these methods.

One skilled in the art will recognize that one route will be optimized depending on the choice of appendage substituents. Additionally, one skilled in the art will recognize that in some cases the order of steps has to be controlled to avoid functional group incompatability.

The prepared compounds may be anyalyzed for their composition and purity as well as characterized by standard analytical techniques such as, for example, elemental anyalysis, NMR, mass spectroscopy and IR spectra.

One skilled in the art will recognize that reagents and solvents actually uised may be selected from several reagents and solvents well known in the art to be effective equivalents. Hence, when a specific solvent or reagent is mentioned, it is meant to be an illustrative example of the conditions deserible for that particular reactionscheme and in the proparations and examples described below.

Where NMR data are presented, ¹H spectra were obtained on either a Varian VXR-200 (200 MHz, ¹H), Varian Gemini-300 (300 MHz), Varian Mercury VX-400 (400MHz), or Bruker-Biospin AV-500 (500MHz), and are reported as ppm with number of protons and multiplicities indicated parenthetically. Where LC/MS data are presented, analyses was performed using an Applied Biosystems API-100 mass spectrometer and C18 column, 10-95% CH₃CN—H₂O (with 0.05% TFA) gradient. The observed parent ion is given.

The following solvents and reagents may be referred to by their abbreviations in parenthesis:
Me=methyl; Et=ethyl; Pr=propyl; Bu=butyl; Ph=phenyl, and Ac=acetyl μl=microliters
AcOEt or EtOAc=ethyl acetate
AcOH or HOAc=acetic acid
ACN=acetonitrile
atm=atmosphere
9-BBN=9-borabicyclo[3.3.1]nonane
Bn=benzyl
Boc or BOC=tert-butoxycarbonyl
Bz=benzoyl
DBU=1,8-Diaza-7-bicyclo[5.4.0]undecene
DCM or CH₂Cl₂: dichloromethane:
DMF=dimethylformamide
DMS=dimethylsulfide
DMSO=dimethyl sulfoxide
dppf=1,2′-bis(diphenylphosphino)ferrocene
EDCI=1-(3-dimethylaminopropyI)-3-ethylcarbodiimide
g=grams
h=hour
LAH=lithium aluminum hydride
LCMS=liquid chromatography mass spectrometry
LDA=Lithium diisopropylamine
min=minute
mCPBA=3-chloroperoxybenzoic acid
mg=milligrams
mL=milliliters
mmol=millimoles
MeOH: methanol
MS=mass spectrometry
NaHMDS=Sodium hexamethyldisilazide
NMO=N-methyl-morpholinoxide
NMR=nuclear magnetic resonance spectroscopy
PTLC=preparative thin layer chromatography
RT or rt=room temperature (ambient, about 25° C.).
TEA or Et₃N=triethylamine
TFA=trifluoroacetic acid
THF=tetrahydrofuran
TLC=thin layer chromatography
TMS=trimethylsilyl
TosMIC=tosylmethyl isocyanide

### EXAMPLES

The compounds of this invention can be prepared through the general approach outlined in the following schemes. These schemes are being provided to illustrate the present invention. Group A is defined in accordance with the definition in the invention; i.e., as a 5-membered heteroaryl, heterocyclyl or heterocyclenyl ring containing 1-3 heteroatoms. The depiction of A as imidazole is not in any way to be considered a limitation of the invention scope. Scheme 1 shows an approach in which S1 is converted to S2 and then further elaborated to S4. In one embodiment, a nitrile may installed by sequential oxidation of S1 (with a reagent such as mCPBA or a peracid to the pyridine N-oxide) and treatment with BzCl/TMSCN to provide compound S2 (Z═CN). When R=Bz (S2a), hydrolysis with base (LiOH, NaOH or the like) is performed to provide S2b. Alternatively, compound S1 is oxidized, treated with an acid halide (BzBr, BzCl, ClCO₂Me, or TMSI/CO₂Me), and then hydroxide base to provide S2b where Z is a halogen (Br, CI, or I). Compound S2b (Z=halogen or nitrile) is converted to S4 by reaction with an electrophilic compound S3 (optionally protected with Boc, trityl or other appropriate group). In various embodiments. R′ is a carboxaldehyde (leading to coupling by reductive amination), a carboxylic acid or acid chloride (leading to amide coupling) or methylene chloride (leading to coupling by alkylation).

In the case where R′ is a carboxylic acid (S3b) or acid chloride (S3d), the carbonyl in resulting amide product may be subsequently reduced to a methylene group. When Z is a halogen in compound S4 or S2, conversion to the nitrile may be accomplished by known metal-catalyzed reactions with zinc, copper, palladium, iron or tin and a cyanide source (including Zn(CN)₂/Pd catalyst; CuI/cyanide source/Pd catalyst; K₄Fe(CN)₆/CuI, nBu₃SnCN/Pd catalyst; CuCN/NaCN and the like). In the case where S2 or S4 is an azaindole system, reduction to the corresponding azaindoline system may be accomplished by known literature methods (such as AcOH/NaBH₃CN, TFA/NaBH₄, TFA/Et₃SiH, or the like). In the case where compound S3 was protected, an appropriate deprotection step is performed (such as treatment with acid when R″=Boc or Tr).

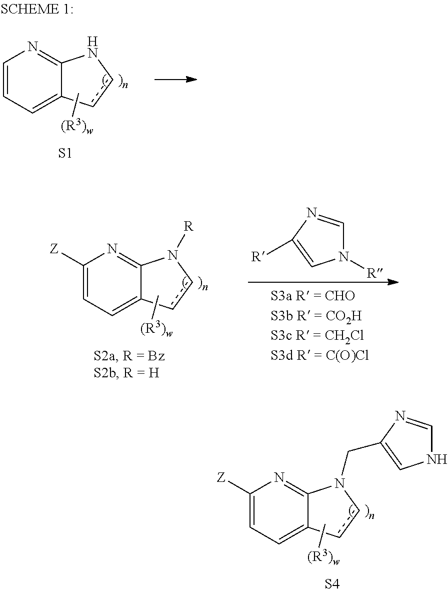

According to another embodiment (Scheme 2), compound S5 (X═NR, O or S) is reacted with an optionally protected S3c or S3d under basic conditions or Lewis acidic conditions to provide S6. In the case where compound S3 was protected, an appropriate deprotection step is performed.

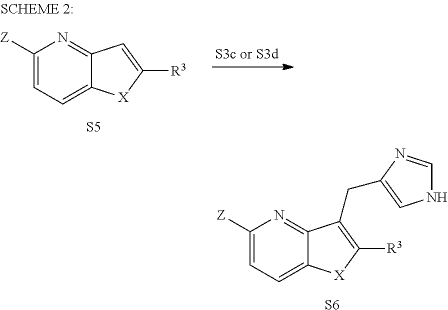

According to another embodiment (Scheme 3), an appropriately substituted quinoline (S7, Z=halogen or methyl; R═H, —CO₂H, or halogen) is converted to S8a or S8b, as described in WO2008100480. When Z=Me, conversion to a nitrile is performed as described in WO 99/41239. When Z=halogen, conversion to a nitrile is done by a metal catalyzed process, as described above in Scheme 1.

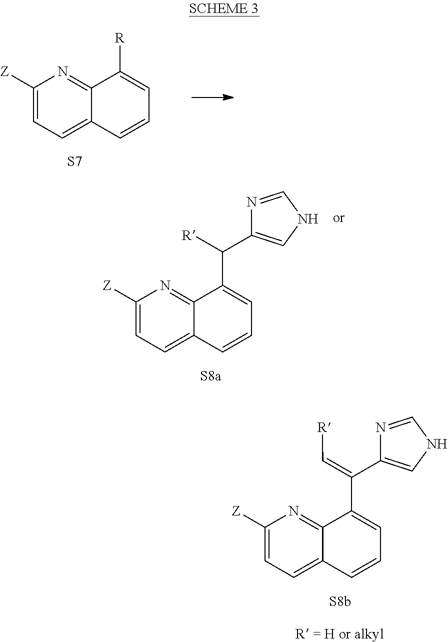

According to another embodiment (Scheme 4), the compound S9 (Z═OH, halogen, or nitrile) is converted to compound S10 (Z═CN or halogen) by known literature methods (see for example J. Org. Chem. 1990, 55, 4789). Further elaboration may be done using a Wittig or Horner-Emmons strategy (see also WO2008100480): Compound S10 is reduced to alcohol S11 by a known method, such as treatment with LiAlH₄, borane, NaBH₄, or the like. The alcohol is then activated and displaced with an appropriate reagent, such as PPh₃HBr or P(OEt)₃, to afford a Wittig or Horner-Emmons type intermediate S12. Subsequent treatment with an appropriate base and an optionally protected imidazole aldehyde S3a under standard Wittig or Horner-Emmons conditions provides S13. The double bond in S13 may be optionally reduced. In the case where compound 3a was protected, a deprotection step is performed.

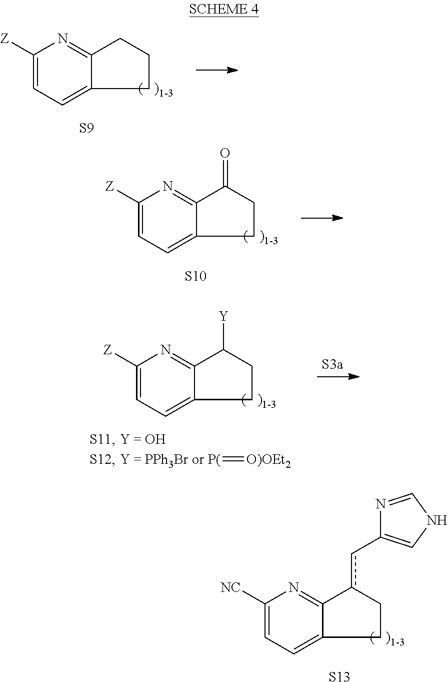

According to another embodiment (Scheme 5), an appropriately substituted pyridine (S14, X═OH, NHR or SH; Z=halogen) is reacted with S15 (wherein Y is an appropriate leaving group, such as halogen, pre-activated alcohol or in-situ activated alcohol) by a displacement reaction. Subsequent intramolecular cyclization of S16 occurs by metal catalysis (such as Pd(0)) or radical cyclization (with nBu₃SnH/AIBN or similar radical promoting reagents). The resulting olefin S17 undergoes an oxidation/cleavage sequence (OsO₄/NaIO₆) to provide a ketone (S18), which is reduced to an alcohol (Y═OH) and optionally converted to a halogen (such as Br by PBr₃ or a similar reagent). The nitrile is then installed via N-oxide formation of S19 (with mCPBA or related peracid) and subsequent reaction with TMSCN/BzCl or the like. The Y group (OH or halogen) in S20a is then displaced with an appropriate reagent, such as PPh₃ or P(OEt)₃, to afford a Wittig or Horner-Emmons type intermediate S20b. Subsequent treatment with an appropriate base and a protected imidazole aldehyde S3a under standard Wittig or Horner-Emmons conditions provides S21. A deprotection step is then performed. The double bond in S21 may be optionally reduced.

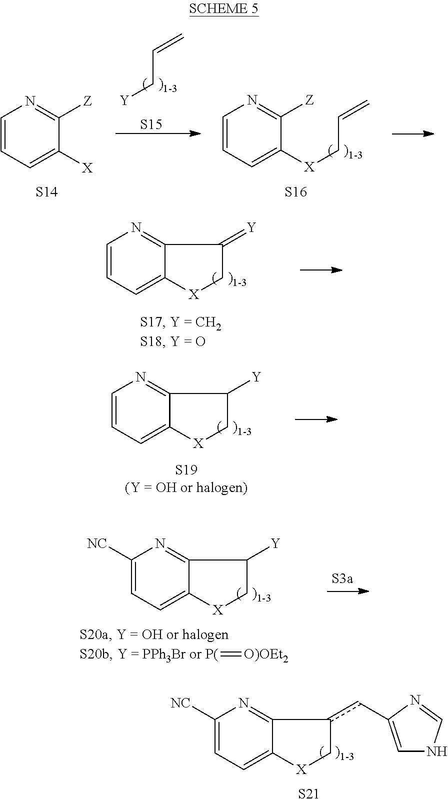

According to another embodiment, the compound S22 (X═O, NR, S or CH₂) is converted to compound S24 as detailed in Scheme 6. Compound S22 is subjected to a two-carbon homologation to yield S23 by one of a number of known methods (see Synthesis, 1979, 633-664, and U.S. Pat. No. 6,841,684). These methods include the sequence of a Wittig or Horner-Emmons (with an appropriate base and a reagent such as diethyl 2,2-diethoxyethylphosphonate or (1,3-dioxolan-2-ylmethyl)tripheneylphosphonium bromide) followed by hydrogenation (with a catalyst such as Pd/C in H₂) and hydrolysis. An alternative method is the sequence of a Wittig or Horner-Emmons (with an appropriate base and a reagent such trimethyl phosphonoacetate or (methoxycarbonylmethyl)triphenylphosphonium bromide) followed by hydrogenation (with a catalyst such as Pd/C in H₂) and transformation (reduction or reduction/oxidation) to the corresponding aldehyde S23. Other alternative methods also include Wittig (with methyl triphenylphosphonium bromide) and hydroboration protocols (with 9-BBN and CO). The compound S23 is then converted to S24 by the sequence of TosMIC/NaCN and then NH₃/MeOH.

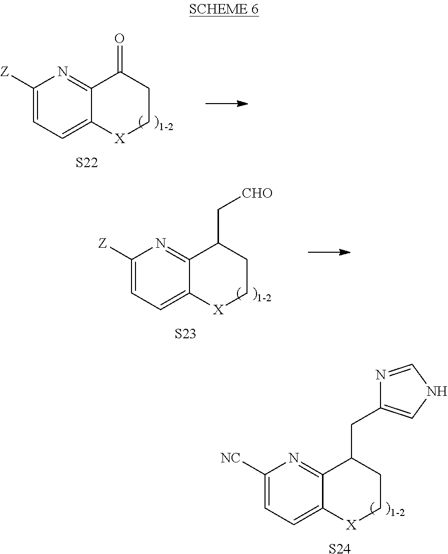

The starting materials and reagents used in preparing compounds described are either available from commercial suppliers such as Aldrich Chemical Co. (Wisconsin, USA) and Acros Organics Co. (New Jersey, USA) or were prepared by literature methods known to those skilled in the art.

Compounds of formulae S4, S6, S8a, S8b, S13, S21, and S24 can be prepared by the general methods outlined above. Exemplary compounds were prepared as described in the examples below or from starting materials known in the art. These examples are being provided to further illustrate the present invention. They are for illustrative purposes only; the scope of the invention is not to be considered limited in any way thereby.

### Preparative Example 1

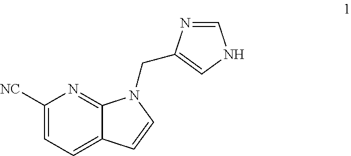

### Steps 1-3

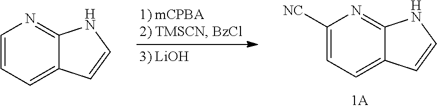

7-azaindoline was sequentially treated with mCPBA (J. Org. Chem. 1980, 45, 4045), TMSCN/benzoyl chloride (Synthesis, 1992, 661) and LiOH (J. Agric. Food Chem. 1997, 45, 2345) to provide 6-cyano-7-azaindole 1A. The formation of compound 1A is also described in the literature by alternate approaches (Synthesis 2008, 201 and Synthesis, 2008, 707).

### Step 4

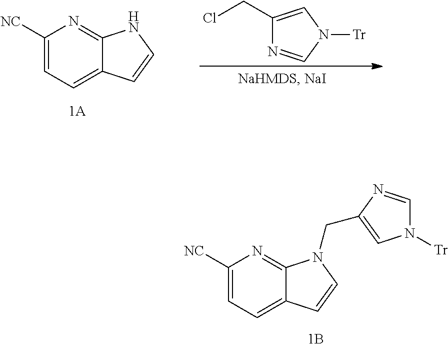

A solution of 1A (45 mg, 0.3 mmol) in THF (5 mL) and DMF (1 mL) was treated with NaH MDS (1 M/THF, 1.5 mL) and stirred at RT for 30 min. NaI (45 mg, 0.3 mmol) and 4-(chloromethyl)-1-tritylimidazole (0.3g, 0.9 mmol) were then added. The reaction was stirred overnight at RT and then concentrated. Chromatography (20-50% EtOAc/hex) provided 1B (72 mg, 50%) and recovered starting material 1A (23 mg, 50%)

### Step 5

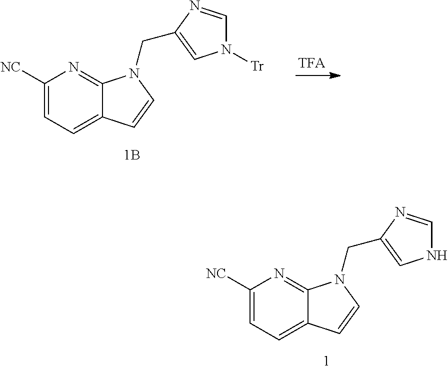

A solution of 1B (70 mg, 0.15 mmol) in DCM (10 mL) was treated with TFA (0.06 mL, 0.75 mmol) and Et₃SiH (0.03 mL, 0.15 mmol). Chromatography (PTLC, 5% of 7N NH₃-MeOH in DCM) provided the title compound 1 (24 mg, 70%). LCMS m/z 224 (MH+).

### Preparative Example 2

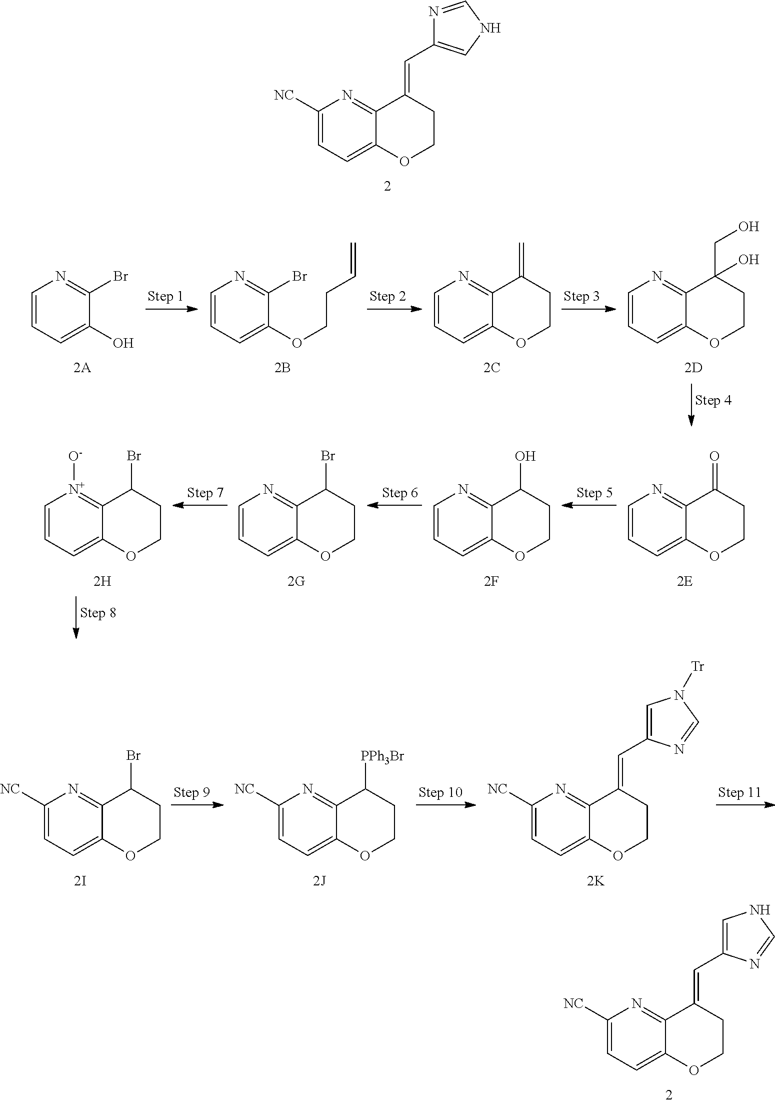

### Step 1

To a stirred mixture of 2-bromo-3-pyridinol (20 g, 115 mmol, 2A) and 3-butenol (10 mL, 117 mmol) in 460 mL of anhydrous THF at 0° C. was added PPh₃ (36.14 g, 138 mmol) followed by diethylazodicarboxylate (19.9 mL, 126.4 mmol). The cooling bath was removed after 10 min. The mixture was heated at reflux over night, and then concentrated in vacuo to dark brown oil. The oil was dissolved in 500 mL of EtOAc, washed with a sat. NaHCO₃ aq. solution and brine, dried with Na₂SO₄, and concentrated in vacuo to a mixture of oil and solid. The crude product mixture was dissolved in ˜120 mL of CH₂Cl₂; the white solid was removed by filtration. The filtrate was purified by flash column chromatography eluting with 10% EtOAc in hexanes to afford 18.76 g of 2B (72%) as a near colorless oil.

### Step 2

A solid mixture of PPh₃ (6.48 g, 24.7 mmol), palladium acetate (1.85 g, 8.22 mmol), potassium acetate (40.36 g, 411.2 mmol), and tetraethylammonium chloride hydrate (27.26 g, 164.5 mmol) in a sealed flask fitted with a septum was degassed via house vacuum, refilled with N₂. A solution of pyridyl bromide 2B (18.76 g, 82.25 mmol) in 330 mL of anhydrous DMF was added. The mixture was degassed again, and refilled with N₂. The septum was quickly replaced by a standard stopper, and the flask was sealed. The mixture was heated at 105° C. over night. After cooling to RT, the mixture was poured into 500 mL of H₂O, extracted with EtOAc (400 mL×3). The combined organic extracts were filtered, and then washed with H₂O then brine. The organic solution was dried over Na₂SO₄, concentrated to a dark brown oil, which was purified by flash column chromatography eluting with 5% EtOAc in hexanes to afford 7.75 g of pyridyl alkene 2C (64%) as a light yellow oil.

### Step 3

To a stirred solution of pyridyl alkene 2C (7.75 g, 52.66 mmol) in 150 mL of CH₂Cl₂ at RT was added NMC) (18.5 g, 158 mmol). A 2.5 wt % solution of osmium tetraoxide in tert-butanol (14 mL, ˜1.05 mmol) was added drop wise. The mixture was stirred overnight, then diluted with 200 mL of EtOAc, and filtered through a celite pad. The filtrate was concentrated in vacuo to a dark brown oil, purified by flash column chromatography, eluting with CH₂Cl₂, CH₂Cl₂-7N NH₃ in MeOH (25:1, v/v) to afford 10.39 g of diol 2D (˜100%) as a yellow oil.

### Step 4

Diol 2D (10.39 g 9.54 g, 52.66 mmol) was dissolved in 100 mL of THF and 100 mL of H₂O. Sodium periodate (33.73 g, 158.0 mmol) was added. The mixture was stirred for 2.5 h at RT. A sat. NaHCO₃aq. solution was added (˜400 mL); the mixture was further diluted with H₂O, and extracted with CH₂Cl₂ (400 mL×3). The organic extracts were filtered through a celite pad, and then washed with HO and brine. The organic solution was dried with Na₂SO₄, and concentrated in vacuo to give 6.02 g of the ketone 2E (77% over two steps) as a light yellow solid.

### Step 5

Sodium borohydride (2.29 g, 60.52 mmol) was added to a stirred solution of ketone 2E (6.02 g, 40.364 mmol) in 200 mL of methanol at RT. The mixture was stirred over night. 200 mL of water was added, stirring was continued for 30 min. The mixture was concentrated in vacua, and extracted with CH₂Cl, (200 mL×3). The combined organic extracts were washed with brine, dried over Na₂SO₄, and concentrated in vacuo to an oil, solidified on standing, providing 5.5 g of alcohol 2F (90%) as a yellow solid.

### Step 6

Phosphorous tribromide (5.2 mL, 55.13 mmoL) was added dropwise to a stirred solution of alcohol 2F (5.5 g, 36.386 mmol) in 200 mL of CHCl₃. The mixture was heated at reflux for 3.5 h, cooled to RT, poured into a mixture of ice and sat. NaHCO₃ aq. solution (˜400 mL, 1 v:1 v). The aqueous mixture was separated and the aqueous layer was extracted with CH₂Cl₂ (300 mL×2). The combined organic extracts were washed with H₂O, sat. NaHCO₃, and brine. The organic solution was dried with Na₂SO₄, filtered, and concentrated in vacuo to give 7.63 g of bromide 2G (98%) as dark pinkish oil.

### Step 7

To a stirred solution of bromide 2G (3.0 g, 14.015 mmol) in 140 mL of CH₂Cl₂ at 0° C. was added mCPBA (3.93 g, 17.535 mmol). The reaction mixture was stirred over night while temperature was increased to RT. A sat. NaHCO₃ aq. solution was added. The layers were separated. The aqueous layer was extracted with CH₂Cl₂ (100 mL×2). The combined organic extracts were washed with a sat. NaHCO₃ aq. solution and brine, dried over Na₂SO₄, and concentrated in vacuo to give 3.34 g of the N-oxide 2H (Q) as a yellow oil.

### Step 8

To a stirred solution of N-oxide 2H (1.26 g, 5.48 mmol) in 18 mL of CH₂Cl₂ at 0° C. was added trimethylsilyl cyanide (4.4 mL, 33 mmol). Benzoyl chloride (1.9 mL, 16.49 mmol) was added dropwise. The mixture was stirred at 0° C. for 30 min, continued at RT for 30 min, and then heated at reflux over 1.5 d. After cooling, a sat. NaHCO₃ aqueous soution was added. Stirring was continued for 2 h. The layers were separated, and the aqueous layer was extracted with CH₂Cl₂ (30 mL×2). The combined organic extracts were washed with brine, dried over Na₂SO₄, filtered, and concentrated in vacuo to a yellow oil, which was purified by column chromatography eluting with CH—Cl₂ in hexanes (10% to 80%) to afford 0.743 g of the nitrile 2I (57%) as a colorless solid.

### Step 9

To a stirred solution of bromide 21(0.743 g, 3.11 mmol) in 20 mL of acetonitrile was added PPh₃ (0.815 g, 3.11 mmol). The mixture was heated at reflux for 1.5 d. Solvent was removed in vacuo, and the oily residue was triturated with ether twice and hexanes once to give 1.56 g of the phosphine salt 2J (100%) as an orange color solid.

### Step 10

To a stirred solution of phosphine salt 2J (1.56 g, 3.11 mmol) in 20 mL of THF at −78° C. was added dropwise a 2.0 M solution of LDA in THF (1.8 mL, 3.6 mmol). The mixture was stirred for 75 min while temperature was maintained at −78° C. to −65° C. A solution of 1-trityl-4-imidazole carboxaldehyde (1.2 g, 3.55 mmol) in 10 mL of THF was added dropwise. Reaction was continued for 2.5 h as temperature of the cooling bath was gradually increased to −20° C. Cooling bath was removed. Reaction was continued for 3 h at RT. 3 mL of methanol was added. After 5 min, the mixture was concentrated in vacuo to a yellow solid, which was purified by column chromatography, eluting with EtOAc in CH₂Cl₂ (0%, 5%, v/v) to afford 220 mg of alkenyl product 2K (15% over two steps) as a light yellow solid.

### Step 11

Alkenyl product 2K (220 mg, 4.58 mmol) was dissolved in 6 mL CH₂Cl₂. Trifluoroacetic acid (0.31 mL, 4.04 mmol) and triethylsilylane (0.13 mL, 0.815 mmol) were added. The mixture was stirred at RT for 1.5 h, and then concentrated in vacuo to a solid residue. This residue was purified directly by column chromatography eluting with MeOH (containing 0.1% triethylamine) CH₂Cl₂ (0%, 5%, 10 to 30%) to afford 108 mg of the title product 2 (99%) as a pale yellow solid. LCMS m/z 239 (MH+).

### Assay:

Efficacy agonist activity values (Emax, GTP₇S assay) for α2A and α2C were determined by following the general procedure detailed by UmLand et. al (“Receptor reserve analysis of the human α_{2c}-adrenoceptor using [³⁵S]GTPγS and cAMP functional assays” European Journal of Pharmacology 2001, 411, 211-221). For the purposes of the present invention, a compound is defined to be a specific or at least selective agonist of the α2C receptor subtype if the compound's efficacy at the α2C receptor is ≧30% Emax (GTPγS assay) and it's efficacy at the α2A receptor is ≦35% Emax (GTPγS assay).

The following compounds were evaluated to be specific or at least selective agonists of the α2C receptor subtype based on the previously defined definition: 1 and 2.

While the present invention has been described with in conjunction with the specific embodiments set forth above, many alternatives, modifications and other variations thereof will be apparent to those of ordinary skill in the art. All such alternatives, modifications and variations are intended to fall within the spirit and scope of the present invention.

1.-11. (canceled)

12. A compound selected from the group consisting of: 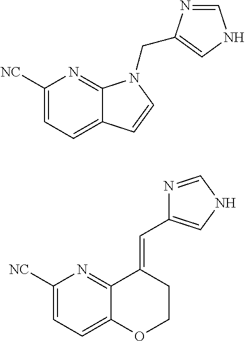
or a pharmaceutically acceptable salt or solvate thereof.

13. A pharmaceutical composition comprising at least one compound according to claim 12, or a pharmaceutically acceptable salt or solvate thereof and at least one pharmaceutically acceptable carrier, adjuvant or vehicle, provided that when the composition is a liquid aqueous composition one or more solubility enhancer components are excluded with the exception of cyclodextrin.

14. The pharmaceutical composition according to claim 13, further comprising one or more additional therapeutic agents.

15. The pharmaceutical composition according to claim 14, wherein said additional therapeutic agents are selected from the group consisting of glucosteroids, PDE-4 inhibitors, anti-muscarinic agents, cromolyn sodium, H₁ receptor antagonists, 5-HT₁ agonists, NSAIDs, angiotensin-converting enzyme inhibitors, angiotensin II receptor agonists, β-blockers, β-agonists, leukotriene antagonists, diuretics, aldosterone antagonists, ionotropic agents, natriuretic peptides, pain management agents, anti-anxiety agents, anti-migraine agents.

16. The pharmaceutical composition according to claim 14, wherein said additional therapeutic agenst are selected from the group consisting of therapeutic agents suitable for treating heart conditions, psychotic disorders, and glaucoma.

17. A method for treating one or more conditions associated with α2C adrenergic receptors, comprising administering to a mannal in need of such treatment a compound according to claim 12 or a pharmaceutically acceptable salt, ester, solvate, or produg thereof.

18. The method according to claim 17, wherein the conditions are selected from the group consisting of allergic rhinitis, congestion, pain, diarrhea, glaucoma, congestive heart failure, cardiac ischemia, manic disorders, depression, anxiety, migraine, stress-induced urinary incontinence, neuronal damage from ischemia, schizophrenia, attention deficit hyperactivity disorder and symptoms of diabetes.

19. The method according to claim 18, wherein the condition is congestion.

20. The method according to claim 19, wherein the congestion is associated with perennial allergic rhinitis, seasonal allergic rhinitis, non-allergic rhinitis, vasomotor rhinitis, rhinitis medicamentosa, sinusitis, acute rhinosinusitis, or chronic rhinosinusitis or the congestion is associated with the common cold.

21. The method according to claim 18, wherein the condition is pain.

22. The method according to claim 21, wherein the pain is associated with neuropathy, inflammation, arthritis, diabetes.
